(19) United States
(12) Patent Application Publication
(10) Pub. No.: US 2011/0179521 A1
(43) Pub. Date: Jul. 21, 2011

(54) PLANT SHOWING RESISTANCE TO MULTIPLE DISEASES AND METHOD FOR PRODUCING THE SAME

(75) Inventors: Yoshihiro Narusaka, Kaga-gun (JP); Mari Narusaka, Kaga-gun (JP); Ken Shirasu, Yokohama-shi (JP)

(73) Assignee: RIKEN, Saitama (JP)

(21) Appl. No.: 13/056,604

(22) PCT Filed: Jul. 29, 2009
(86) PCT No.: PCT/JP2009/063474

§ 371 Date: Mar. 24, 2011

(30) Foreign Application Priority Data
Jul. 31, 2008 (JP) 2008-198517

(51) Int. Cl. A01H 1/06; A01H 5/00

(52) U.S. Cl. 800/279; 800/301

(57) ABSTRACT

It is found that multiple different resistance genes adjacent to each other on the genome of Arabidopsis recognize attacks from multiple different pathogens and activate the resistance reactions. Introduction of a combination of multiple different resistance genes into a plant makes it possible to provide the plant with resistance to multiple different pathogens.

### TECHNICAL FIELD

The present invention relates to a method for providing a plant with resistance to two or more pathogens by introducing a combination of two or more resistance genes to the pathogens into the plant; a transgenic plant produced by the method; and a portion thereof or a propagation material.

### BACKGROUND ART

It is said that 10 to 20% of agricultural production worldwide suffers from diseases. This corresponds to foods for 800 million people. Since the world starving population is estimated to be 800 million, to prevent such diseases is the most important issue for stable food supply. The factor of causing diseases in plants includes an infectious biological agent (pathogen) and a non-infectious environmental agent. Filamentous fungi (molds, fungi) cause 80% or more of plant infectious diseases, whereas the rest of the plant infectious diseases are caused by bacteria, viruses, viroids, phytoplasma, rickettsia-like microorganisms, nematodes, protists, and the like. Plants are always exposed to attacks from these pathogens, and protect themselves from the pathogenic infection by own biological defense reactions.

For example, Cruciferous vegetable anthracnose is a disease caused by a filamentous fungus (Colletotrichum higginsianum) in the genus Colletotrichum as a pathogen. Cruciferous crops such as Japanese mustard spinach, Chinese cabbage and white radish are infected with and damaged by the disease. Recently, a number of Arabidopsis ecotypes have been found which show resistance to this pathogen (Non Patent Literature 1). Moreover, the present inventors have found that a gene in a specific region on chromosome 5 of the resistant ecotype Ws-0 has a function to provide Arabidopsis with resistance to Cruciferous vegetable anthracnose.

In addition, Arabidopsis has been discovered to have a resistance gene to a pathogen of bacterial leaf spot of tomato (pathogenic bacterium Pseudomonas syringae pv. tomato) which infects mainly Solanaceae plants and which has an avirulence gene avrRps4 (Non Patent Literature 2). Furthermore, Arabidopsis has also been discovered to have a resistance gene to a bacterial wilt (Ralstonia solanacearum) which infects and blights over 200 species of plants such as Solanaceae and Cruciferous plants and which causes detrimental damage in agriculture (Non Patent Literatures 3 to 5).

Such biological defense reactions of plants are thought, according to the gene-for-gene hypothesis proposed by Flor (Non Patent Literature 6), to take place when a resistance gene product of a plant recognizes an elicitor (avirulence gene product) derived from a pathogen and expresses a series of resistance reactions to the pathogen. In addition, in this hypothesis, one resistance gene acts on one pathogen on a one-for-one basis.

Although hundreds of thousands of filamentous fungi exist on the earth, the majority thereof do not infect plants. Only approximately 8000 species are plant pathogens, among which no more than 10 species cause severe damages to single plant species. In other words, plants show resistance to most of the pathogens. Despite this, only approximately 150 (Non Patent Literature 7) and approximately 600 (Non Patent Literature 8) resistance genes are present on the Arabidopsis genome and on the rice genome, respectively. Accordingly, plant resistance mechanisms to various pathogens cannot be explained only by the gene-for-gene hypothesis. To date, the details thereof have not been elucidated yet.

### CITATION LIST

### Non Patent Literatures

[NPL 1] Y. Narusaka et al. Molecular Plant-Microbe Interactions, 17: 749-762 (2004)
[NPL 2] W. Gassmann et al. Plant J. 20: 265-277 (1999)
[NPL 3] L. Deslandes et al. Molecular Plant-Microbe Interactions, 11 (7): 659-667 (1998)
[NPL 4] L. Deslandes et al. Proc Natl Acad Sci USA., 99 (4): 2404-2409 (2002)
[NPL 5] L. Deslandes et al. Proc Natl Acad Sci USA., 24: 8024-8029 (2003)
[NPL 6] H. Flor. Annu. Rev. Phytopathol. 9: 275-296 (1971)
[NPL 7] Meyers, B. C. et al. Plant Cell, 15: 809-834 (2003)
[NPL 8] Goff, S. A. et al., Science, 296: 92-100 (2002)
[NPL 9] Tsuchiya et al., Plant Protection 54:87-92 (2000)
[NPL 10] Clark et al., Science, 317: 338-342 (2007)
[NPL 11] Fedoroff N V. and Smith D. Plant J. 3: 273-289 (1993)
[NPL 12] Noutoshi et al. Plant J., 43: 873-888 (2005)

### SUMMARY OF INVENTION

### Technical Problem

The present invention has been made in view of such circumstances. An object thereof is to elucidate a mechanism in plant resistance to various pathogens, and to produce a plant showing resistance to various pathogens by utilizing the mechanism.

### Solution to Problem

The present inventors have revealed for the first time in the world that a Cruciferous vegetable anthracnose pathogen (Colletotrichum higginsianum) infects the Arabidopsis ecotype Col-0, and established a model experimental system for plant immunology research (Non Patent Literature 1). As a result of infection-physiological and genetic analyses on over 100 Arabidopsis ecotypes, 11 ecotypes showing resistance to this pathogen are obtained. Then, the inventors have revealed that the resistance of Eil-0 showing particularly strong resistance among these is dominant and controlled at a single locus, and this gene is present on chromosome 4 among five chromosomes of Arabidopsis (Non Patent Literature 1). The gene (locus) is highly likely to be a resistance gene (R-gene, a receptor on the plant side that recognizes the pathogen), and is named as RCH1 (for Recognition of C. higginsianum).

It has been suggested that, in other resistant ecotypes, a resistance gene is present at a locus different from that in Eil-0. Accordingly, the present inventors have proceeded with the analysis on this gene and found out that the resistance gene in the ecotype Ws-0 is present on chromosome 5. As a result of detailed mapping of the resistance gene in the ecotype Ws-0, the target gene is located in a region where a cluster of over ten resistance genes is formed (MRC-J-region). Then, the present inventors have screened for mutants whose phenotype changed from resistant to susceptible to an anthracnose pathogen through the reverse genetic analysis using a mutant library where a T-DNA is inserted into the ecotype Ws-0 (30,000 individuals). The analysis on the T-DNA insertion sites and mutation sites of eight mutants thus obtained has revealed that the susceptible mutants have a mutation in Ws-At5g45250 (designated as RPS4 by Gassmann) or Ws-At5g45260 gene (designated as RCH2 by the present inventors).

Several reports have been made so far on the relationships between RPS4, RCH2 and the resistance to pathogen. For example, Gassmann et al. have already identified that RPS4 in the Arabidopsis ecotypes Col-0 and Ler-0 is a resistance gene to a pathogen of bacterial leaf spot of tomato (pathogenic bacterium Pseudomonas syringae pv. tomato) (Non Patent Literature 2). Meanwhile, Gassmann et al. states that RCH2 (in the literature, referred to as RSH4) present adjacent to RPS4 has nothing to with the pathogen of bacterial leaf spot of tomato (Non Patent Literature 2).

Moreover, a different research group than Gassmann et al. has identified that RCH2 (in the literature, referred to as RRS1-R) in the Arabidopsis ecotypes Nd-1 and Ct-1 is a resistance gene to a bacterial wilt pathogen (soilborne pathogenic bacterium Ralstonia solanacearum) which infects Solanaceae and Cruciferous plants (Non Patent Literatures 3 to 5). Meanwhile, this research group states that RPS4 has nothing to do with the resistance to the bacterial wilt pathogen (Non Patent Literature 4).

As described above, RPS4 and RCH2 have been heretofore identified as the resistance genes to pathogens other than the anthracnose pathogen. However, the present inventors have revealed that the genes function as the resistance genes to the anthracnose pathogen.

Furthermore, as a result of analyzing the relationship between the resistance/susceptibility and the two genes in various ecotypes, the present inventors have revealed that both RPS4 and RCH2 are necessary to induce the resistance to the pathogens of anthracnose, bacterial leaf spot of tomato, and bacterial wilt.

This result demolishes the conventional reports that RPS4 and RCH2 respectively have nothing to do with the pathogens of bacterial wilt and bacterial leaf spot of tomato, and also changes the conventional concept on the mechanism in the resistance reactions of plants to pathogens.

Specifically, it has been thought, according to the gene-for-gene hypothesis proposed by Flor, that the resistant reactions of plants to pathogens are determined by a 1:1 combination of a resistance gene of a plant with a corresponding avirulence gene (Avr-gene) of a pathogen (Non Patent Literature 6). However, the present inventors have discovered for the first time in the world that two different resistance genes adjacent to each other on the genome of Arabidopsis recognize attacks from three different pathogens and activate the resistance reactions. The present invention has reveals that the immune system of plants, as similar to that of animals, recognizes various pathogens by combining a few genes and expresses the defense system.

In other words, the present invention is based on the discovery of the novel mechanism in the resistance reactions of plants to pathogens. More specifically, the present invention is as follows.

<1> A method for providing a plant with resistance to two or more pathogens, the method characterized by comprising: introducing a combination of two or more genes into the plant, wherein the genes do not provide the plant with the resistance to the two or more pathogens when each of the genes is introduced alone, but provide the plant with the resistance to the pathogens when introduced in the combination.
<2> The method according to <1>, wherein the pathogens are pathogens of Cruciferous vegetable anthracnose and bacterial leaf spot of tomato, and the combination of the genes is a combination of an RPS4 gene and an RCH2 gene.
<3> The method according to <2>, wherein the RPS4 gene and the RCH2 gene are derived from an Arabidopsis ecotype selected from the group consisting of Ws-0, No-0, Nd-1, Aa-0, Eil-0, Rrs-7, Sha, Tamm-2, Tsu-1, Fei-0, Ts-1, Bsch-0, Br-0, Est-1, Rrs-10, Van-0, Nfa-8, and Bay-0.
<4> The method according to any one of <2> and <3>, wherein the pathogens further include a bacterial wilt pathogen.
<5> A transgenic plant provided with resistance to two or more pathogens, comprising: a combination of two or more genes introduced therein, wherein the genes do not provide the plant with the resistance to the two or more pathogens when each of the genes is introduced alone, but provide the plant with the resistance to the pathogens when introduced in the combination.
<6> The transgenic plant according to <5>, wherein the pathogens are pathogens of Cruciferous vegetable anthracnose and bacterial leaf spot of tomato, and the combination of the genes is a combination of an RPS4 gene and an RCH2 gene.
<7> The transgenic plant according to <6>, wherein the RPS4 gene and the RCH2 gene are derived from an Arabidopsis ecotype selected from the group consisting of Ws-0, No-0, Nd-1, Aa-0, Eil-0, Rrs-7, Sha, Tamm-2, Tsu-1, Fei-0, Ts-1, Bsch-0, Br-0, Est-1, Rrs-10, Van-0, Nfa-8, and Bay-0.
<8> The transgenic plant according to any one of <6> and <7>, which further has resistance to a bacterial wilt pathogen.
<9> A transgenic plant which is any one of a progeny and a clone of the transgenic plant according to any one of <5> to <8>.
<10> Any one of a portion and a propagation material of the transgenic plant according to any one of <5> to <9>.

### Advantageous Effects of Invention

According to the present invention, a combination of multiple different resistance genes is introduced to a plant, enabling the plant to recognize attacks from multiple different pathogens and to activate the resistance reaction. Utilization of the mechanism of the resistance in plants to diseases discovered by the present inventors makes it possible to produce plants showing resistance to a wide range of diseases. For example, by introducing the diseases resistance genes RPS4 and RCH2 into a plant simultaneously, it is possible to provide the plant with resistance to pathogens of Cruciferous vegetable anthracnose, bacterial leaf spot, and bacterial wilt. Thereby, crops are protected from diseases due to the pathogens of Cruciferous vegetable anthracnose, bacterial leaf spot, and bacterial wilt, thus improving the productivity of Cruciferous crops, Solanaceae crops, and the like. Moreover, cases are reported that, when a disease resistance gene is introduced solely, the plant is generally dwarfed. Nevertheless, the combination of multiple genes of the present invention can avoid such a problem of dwarfed plants in producing resistant plants.

### BRIEF DESCRIPTION OF DRAWINGS

FIG. 1 is a drawing showing a region where resistance genes exist, the region being narrowed down by an SSLP (simple sequence length polymorphism) analysis. In the Arabidopsis ecotype (Ws-0), resistance genes At5g45250 and At5g45260 are present adjacent to each other. CIW9, N01-K9E15, nga129, N01-MLN1, N01-K17O22, and N01-K₁₁I1 indicate SSLP markers.

FIG. 2 is a drawing showing mutants of the Arabidopsis ecotypes Ws-0 and Col-0 as well as No-0 from Ms. Fedoroff.

FIG. 3 is a drawing showing comparison of the At5g45260 amino acid sequence between the Arabidopsis ecotypes Ws-0 and Col-0. From At5g45260 of Col-0, 83 amino acids at a portion indicated by the arrow in the drawing are deleted as compared with Ws-0.

FIG. 4A is a graph showing the result of analyzing the relationship between a mutation in At5g45260 and resistance to bacterial leaf spot, illustrating the influence of disruption of At5g45260 (RCH2) in the ecotype Ws-0 resistant to an anthracnose pathogen. In the graph, each of Ws-0, Col-0, and RLD-0 indicates a wildtype. In addition, “rch2-2” (ΔAt5g45260-2) and “rch2-1” (ΔAt5g45260-1) indicate disruptants (2 cases) of At5g45260 in Ws.

FIG. 4B is a graph showing the result of analyzing the relationship between a mutation in At5g45250 and resistance to bacterial leaf spot, illustrating the influence of disruption of At5g45250 (RPS4) in the ecotype Ws-0 resistant to the anthracnose pathogen and the influence of introduction of At5g45250 in the ecotype RLD. In the graph, each of Ws-0, Col-0, and RLD-0 indicates a wildtype. Moreover, “rps4-21” (ΔAt5g45250-21) indicates a disruptant of Ws-At5g45250; “rps4-21-C9” and “rps4-21-C11” indicate revertants (2 cases) obtained by reintroducing Ws-At5g45250 into disruptants of Ws-At5g45250; and “RLD/RPS4-Ws-2” and “RLD/RPS4-Ws-3” indicate transformants (2 cases) obtained by introducing Ws-At5g45250 into RLD-0.

FIGS. 5A to G are pictures showing the result (morphology of organisms) of analyzing the relationship between mutations in At5g45250 and At5g45260 and resistance to bacterial wilt. In the pictures, Nd-1 (resistant), Ws-0 (moderately resistant), No-0 (resistant), and Col-0 (susceptible) indicate wildtypes. rps4-31 (ΔAt5g45250-31) is a mutant of No-0 from Ms. Fedoroff having At5g45250 disrupted, and is susceptible to a bacterial wilt pathogen. rps4-21 (ΔAt5g45250-21) is a mutant of Ws-0 having At5g45250 disrupted, and is susceptible to the bacterial wilt pathogen. rch2-1 (ΔAt5g45260-1) is a mutant of Ws-0 having At5g45260 disrupted, and is susceptible to the bacterial wilt pathogen.

FIGS. 6A to 6D are pictures showing the result (morphology of organisms) of introducing a resistance gene into the Arabidopsis ecotypes Col-0 and RLD-0 susceptible to the anthracnose pathogen. Ws-At5g45250/Col-0 is a transformant obtained by introducing the resistance gene Ws-At5g45250 into the wildtype Col-0. Ws-At5g45250/RLD is a transformant obtained by introducing the resistance gene Ws-At5g45250 into the wildtype RLD-0. No-0-At5g45260/Col-0 is a transformant obtained by introducing a resistance gene No-0-At5g45260 from Ms. Fedoroff into the wildtype Col-0.

FIG. 7 shows the result of a phylogenetic tree analysis conducted on the amino acid sequences of At5g45250 and At5g45260 by utilizing data analyzed by the present inventors and SNP analysis data on Arabidopsis (Non Patent Literature 10).

FIG. 8 is a continuation of FIG. 7.

FIG. 9 shows comparison of the RPS4 (At5g45250) amino acid sequence among ecotypes. The table shows amino acids not conserved among the ecotypes. The 950th tyrosine of RLD-0 susceptible to all of Cruciferous vegetable anthracnose, bacterial leaf spot, and bacterial wilt is substituted with histidine.

FIG. 10 shows the comparison of the RCH2 (At5g45260) amino acid sequence among ecotypes. In the region of the 1st to 1290th amino acids, amino acids not conserved among the ecotypes are shown based on the amino acid sequence of Col-0. In the region of the 1291th to 1379th amino acids, amino acids not conserved among the ecotypes are shown. The 775th tyrosine of the ecotypes (Ler-1, Lov-5, C24) susceptible to the anthracnose pathogen is substituted with asparagine. Approximately 80 to 90 amino acids are deleted from the WRKY domain of Col-0 toward the C terminal (FIG. 3). The ecotypes Cvi-0 and Bur-0 susceptible to the anthracnose pathogen are also recognized to have deletion toward the C terminal as in Col-At5g45260.

FIG. 11 is a continuation of FIG. 10.

FIG. 12 is a picture showing the result (morphology of organisms) of introduction of resistance genes into the Arabidopsis ecotype Col-0 susceptible to the anthracnose pathogen. Ws-At5g45250/Col-0 is a transformant obtained by introducing the resistance gene Ws-At5g45250 into the wildtype Col-0. Ws-At5g45250•At5g45260/Col-0 is a transformant obtained by introducing the resistance gene Ws-At5g45260 into Ws-At5g45250/Col-0.

FIG. 13 is a picture showing the symptom (morphology of an organism) six days after the anthracnose pathogen is sprayed on and inoculated into Ws-At5g45250•At5g45260/Col-0 shown in FIG. 12.

FIG. 14 is pictures showing the result (morphology of organisms) of introduction of resistance genes into an anthracnose-pathogen susceptible cultivar “Osome” of Cruciferous crop Japanese mustard spinach. Ws-At5g45260/Japanese mustard spinach is a transformant obtained by introducing the Arabidopsis resistance gene Ws-At5g45260 into the wildtype Japanese mustard spinach. Ws-At5g45250•At5g45260/Japanese mustard spinach is a transformant obtained by introducing the Arabidopsis resistance genes Ws-At5g45250 and Ws-At5g45260 into the wildtype Japanese mustard spinach at the same time. Shown are the lesions six days after the anthracnose pathogen is sprayed on and inoculated into the wildtype Japanese mustard spinach and the transformed Japanese mustard spinach.

### DESCRIPTION OF EMBODIMENTS

The present invention is based on an innovative finding in resistance reactions of plants to pathogens that a combination of multiple different resistance genes allows a plant to recognize attacks from multiple different pathogens and to activate the resistance reactions. Accordingly, a method of the present invention is a method for providing a plant with resistance to two or more pathogens, the method characterized by comprising: introducing a combination of two or more genes into the plant, wherein the genes do not provide the plant with sufficient resistance to these pathogens when each of the genes is introduced alone, but provide the plant with these resistance to the pathogens when introduced in the combination.

In the present invention, a “combination of genes that provides a plant with a resistance to two or more pathogens” is not limited, as long as the combination is capable of activating the resistance reactions of the plant to the pathogens in accordance with the above-described mechanisms. For example, in a case where the pathogens are pathogens of Cruciferous vegetable anthracnose and bacterial leaf spot of tomato, the combination is a combination of an RPS4 gene and an RCH2 gene. Herein, “Cruciferous vegetable anthracnose” is a disease caused by a mold (filamentous fungus) called Colletotrichum higginsianum as a pathogen. The disease occurs in Cruciferous crops such as Japanese mustard spinach, turnip, Chinese cabbage, and white radish. Meanwhile, “bacterial leaf spot of tomato” is a disease caused by a bacterium called Pseudomonas syringae pv. tomato as a pathogen. The disease mainly occurs in Solanaceae plants and Arabidopsis. Examples of the pathogens which are targets of the resistance provided to a plant by utilizing the combination of the RPS4 gene and the RCH2 gene further include a bacterial wilt pathogen. “Bacterial wilt” is a disease caused by a bacterium called Ralstonia solanacearum as a pathogen. Over 200 plants including Solanaceae crops such as eggplant and tomato, Cruciferous crops such as white radish, and strawberry are infected with and damaged by the disease. In our country, it is known that this disease has occurred in plants in 38 species in 20 families so far (Non Patent Literature 9).

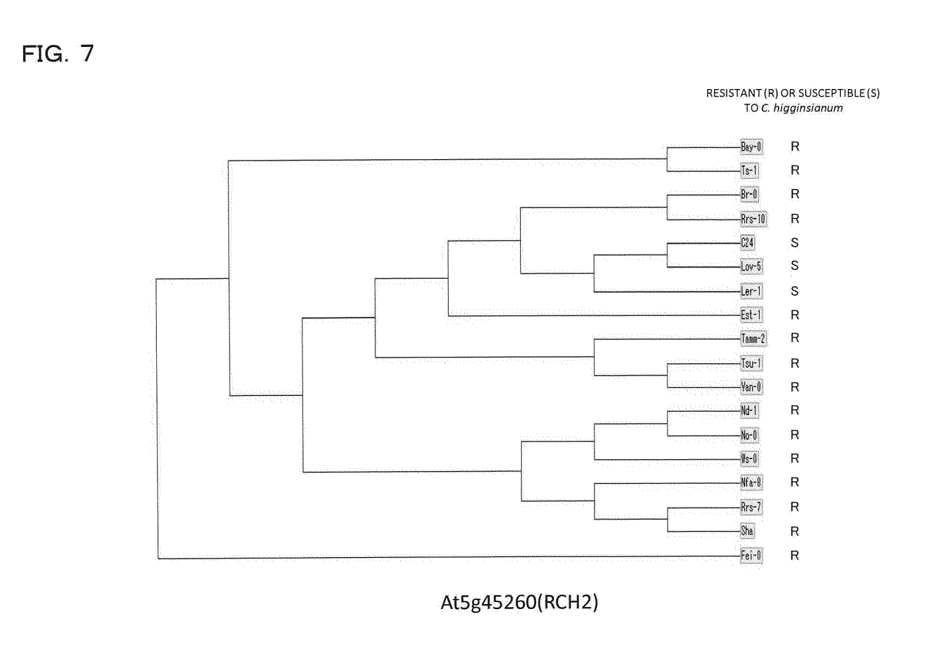

The RPS4 gene and the RCH2 gene used in the present invention are not particularly limited, as long as the genes are derived from a plant which is an ecotype resistant to the pathogens of Cruciferous vegetable anthracnose and bacterial leaf spot of tomato. Preferred examples of Arabidopsis ecotypes from which the RPS4 gene and the RCH2 gene are derived include Ws-0, No-0, Nd-1, Aa-0, Eil-0, Rrs-7, Sha, Tamm-2, Tsu-1, Fei-0, Ts-1, Bsch-0, Br-0, Est-1, Rrs-10, Van-0, Nfa-8, and Bay-0 (see FIGS. 7 to 11). The RPS4 gene and the RCH2 gene of Arabidopsis are respectively provided with AGI codes, At5g45250 and At5g45260. Specific sequences of these can be extracted from Non Patent Literature 10 or the site of POLYMOLPH: “http://polymorph.weigelworld.org/”. Note that, regarding the RPS4 gene, the base sequence of a DNA derived from the Arabidopsis ecotype Ws-0 is represented by SEQ ID NO: 1, and the amino acid sequence of a protein thereof is represented by SEQ ID NO: 2; the base sequence of a DNA derived from the Arabidopsis ecotype No-0 (Ms. Fedoroff: Non Patent Literature 11) is represented by SEQ ID NO: 5, and the amino acid sequence of a protein thereof is represented by SEQ ID NO: 6; and the base sequence of a DNA derived from the Arabidopsis ecotype Nd-1 is represented by SEQ ID NO: 9, and the amino acid sequence of a protein thereof is represented by SEQ ID NO: 10. Regarding the RCH2 gene, the base sequence of a DNA derived from the Arabidopsis ecotype Ws-0 is represented by SEQ ID NO: 3, and the amino acid sequence of a protein thereof is represented by SEQ ID NO: 4; the base sequence of a DNA derived from the Arabidopsis ecotype No-0 (Ms. Fedoroff: Non Patent Literature 11) is represented by SEQ ID NO: 7, and the amino acid sequence of a protein thereof is represented by SEQ ID NO: 8; and the base sequence of a DNA derived from the Arabidopsis ecotype Nd-1 is represented by SEQ ID NO: 11, and the amino acid sequence of a protein thereof is represented by SEQ ID NO: 12.

Particularly, when the resistance to bacterial wilt is provided to a plant, a combination of the RPS4 gene and the RCH2 gene derived from the Arabidopsis resistant ecotypes Nd-1, No-0 (Ms. Fedoroff: Non Patent Literature 11), and moderately resistant Ws-0 is particularly preferably used.

Furthermore, a DNA (for example, mutants in the RPS4 gene and the RCH2 gene, or homologs in plants other than Arabidopsis) encoding a protein structurally similar to the protein encoded by the RPS4 gene or the RCH2 gene derived from Arabidopsis can also be used in the present invention. Examples of the DNA encoding a protein structurally similar to a protein encoded by the RPS4 gene or the RCH2 gene include (i) a DNA encoding a protein having an amino acid sequence in which one or multiple amino acids are substituted, deleted, inserted, and/or added in the amino acid sequence of the protein encoded by the RPS4 gene or the RCH2 gene derived from an Arabidopsis line of an ecotype resistant to the pathogens of Cruciferous vegetable anthracnose and bacterial leaf spot of tomato, (ii) a DNA which hybridizes under stringent conditions to a DNA having the base sequence of the RPS4 gene or the RCH2 gene derived from an Arabidopsis line of an ecotype resistant to the pathogens of Cruciferous vegetable anthracnose and bacterial leaf spot of tomato, and (iii) a DNA encoding a protein having an amino acid sequence having 90% or higher homology to the amino acid sequence of the protein encoded by the RPS4 gene or the RCH2 gene derived from an Arabidopsis line of an ecotype resistant to the pathogens of Cruciferous vegetable anthracnose and bacterial leaf spot of tomato. The combination of the RPS4 gene or the DNA encoding a protein structurally similar to a protein encoded by the RPS4 gene with the RCH2 gene or the DNA encoding a protein structurally similar to a protein encoded by the RCH2 gene is not particularly limited, as long as the introduction of the combination provides a plant with resistance to at least the pathogens of Cruciferous vegetable anthracnose and bacterial leaf spot of tomato.

Herein, “multiple amino acids” subjected to the substitution or the like are generally in a range from 1 to 30 amino acids, preferably from 1 to 10 amino acids, more preferably from 1 to 5 amino acids, and further preferably from 1 to 3 amino acids. The region where such amino acids may be substituted, deleted, added and/or inserted is also not particularly limited, as long as the above resistance is maintained. The “stringent conditions” refer to a sodium concentration of 25 to 500 mM, preferably 25 to 300 mM, and a temperature of 42 to 68° C., preferably 42 to 65° C. For example, the concentration is 5×SSC (83 mM NaCl, 83 mM sodium citrate), and the temperature is 42° C. The “90% or higher homology” indicates a sequence homology of 90% or higher, preferably 95% or higher (for example, 96% or higher, 97% or higher, 98% or higher, and 99% or higher) to the entire amino acid sequence. The homology of the amino acid sequence or the base sequence can be determined by using an algorithm BLAST by Karlin and Altschul (Proc. Natl. Acad. Sci. USA, 1990, 87, 2264-2268., Karlin, S. & Altschul, S F., Proc. Natl. Acad. Sci. USA, 1993, 90, 5873). Programs called BLASTN and BLASTX based on BLAST algorithm have been developed (Altschul, S F. et al., J Mol Biol, 1990, 215, 403). When the base sequence is analyzed using BLASTN, the parameters are set at, for example, score=100 and wordlength=12. Alternatively, when the amino acid sequence is analyzed using BLASTX, the parameters are set at, for example, score=50 and wordlength=3. When BLAST and Gapped BLAST programs are used, the default parameters of each program are used. The specific procedures of these analysis methods are known (http://www.ncbi.nlm.nih.gov/).

The DNA encoding a protein structurally similar to the protein encoded by the RPS4 gene or the RCH2 gene can be prepared by methods well-known to those skilled in the art, for example, hybridization techniques (Southern, E M., J Mol Biol, 1975, 98, 503), polymerase chain reaction (PCR) techniques (Saiki, R K. et al., Science, 1985, 230, 1350., Saiki, R K. et al., Science, 1988, 239, 487), or a method in which a mutation is introduced into the DNA by site-directed mutagenesis (Kramer, W. & Fritz, H J., Methods Enzymol, 1987, 154, 350). Moreover, in nature also, a mutation in the amino acid sequence of the protein to be encoded may occur by a mutation at the base sequence. Examples of the form of these DNAs include genomic DNA, cDNA, and chemically synthesized DNA, which can be prepared by using the conventional methods.

Whether or not the combination of the DNAs thus prepared provides a plant with the resistance to the pathogens can be assessed as follows. Specifically, a transgenic plant in which the combination of these DNAs is introduced is prepared, and it is examined whether or not the occurrence of a disease is suppressed in such a state that the transgenic plant is exposed to a disease stress.

The preparation of the transgenic plant can be carried out, for example, by inserting the combination of the DNAs of the present invention into a vector which guarantees the expression in a plant, and then causing expression of the combination of the DNAs of the present invention in the plant by utilizing the vector. A promoter used in the vector is not limited to a dedicated promoter which controls the expression of the DNAs of the present invention in a natural condition. For example, it is possible to use a CaMV35S promoter which is derived from cauliflower mosaic virus and widely used for plants. The target of the transformation may be a whole plant, a plant organ (for example, seed, leaf, petal, stem, root, or the like), a plant tissue (for example, epidermis, phloem, parenchyma, xylem, vascular bundle, or the like), or a plant cultured cell. The plant used for the transformation is, according to the object of the present invention, suitably a plant (ecotype) susceptible to pathogens of Cruciferous vegetable anthracnose and bacterial leaf spot of tomato. Examples of the plant species include Cruciferous plants such as Arabidopsis (Arabidopsis thaliana), Chinese cabbage, Japanese mustard spinach, turnip (hereinabove, Brassica rapa), cabbage (Brassica oleracea), rapeseed (Brassica napus), and white radish (Raphanus sativus), Solanaceae plants such as tomato (Solanum lycopersicum L.), eggplant (Solanum melongena), bell pepper (Capsicum annuum cv. Grossum), potato (Solanum tuberosum), and tobacco (Nicotiana tabacum), chrysanthemum (Chrysanthemum morifolium) in the family Asteraceae, banana (Musa spp., Musa acuminata, Musa balbisiana) in the family Musaceae, strawberry (Fragaria ananassa) in the family Rosaceae, bitter melon (Momordica charantia L. var. pavel Crantz) in the family Cucurbitaceae, and the like, but not limited to these exemplified plants.

The expression vector can be introduced into the plant by generally-used transformation processes, for example, an electroporation method, an agrobacterium method, a particle gun method, a PEG method, and the like. For example, when the electroporation method is used, a sample is treated at a voltage of 500 to 1600 V under 25 to 1000 μF and 20 to 30 msec with an electroporator equipped with a pulse controller, and thus the genes are introduced into the host. Meanwhile, when the particle gun method is used, the whole plant, the plant organ or the plant tissue itself may be used directly, or a segment thereof may be prepared and then used. Alternatively, a protoplast thereof may be prepared and used. The sample thus prepared can be treated using a gene delivery system (for example, PDS-1000/He manufactured by Bio-Rad Laboratories, Inc., or the like). The treatment conditions vary depending on the plant or sample, but generally the treatment is performed at a pressure of approximately 1000 to 1800 psi and a distance of approximately 5 to 6 cm.

Moreover, using a plant virus as the vector, the DNAs of the present invention can be introduced into the plant. An example of the plant virus utilizable includes cauliflower mosaic virus. Specifically, first, the virus genome is inserted into a vector derived from Escherichia coli, or the like to prepare a recombinant. Then, these target DNAs are introduced into the virus genome. The virus genome thus modified is cut from the recombinant with a restriction enzyme and inoculated into the plant host. Thereby, the target DNAs can be introduced into the plant host.

In a method in which a Ti plasmid of Agrobacterium is utilized, the target DNA can be introduced into the plant host by utilizing the ability that, when a bacterium in the genus Agrobacterium infects a plant, part of the plasmid DNA the bacterium has is transferred into the plant genome. Among bacteria in the genus Agrobacterium, Agrobacterium tumefaciens infects a plant and forms tumors called crown galls, while Agrobacterium rhizogenes infects and causes a plant to form hairy roots. These are based on the ability: at the infection, a region called a T-DNA region (Transferred DNAs) on a plasmid, called a Ti plasmid or an Ri plasmid, present in each bacterium is transferred into a plant and incorporated into the genome of the plant. If DNAs desired to be incorporated into the plant genome are inserted in the T-DNA region on the Ti or Ri plasmid, the target DNAs can be incorporated into the plant genome when the bacterium in the genus Agrobacterium infects the plant host.

The tumor tissue, shoot, hairy root, or the like obtained as a result of the transformation can be used directly for the cell culture, tissue culture or organ culture. Furthermore, using conventionally known plant tissue culture techniques, the tumor tissue or the like can be regenerated to a whole plant by, for example, administration of a plant hormone (auxin, cytokinin, gibberellin, abscisic acid, ethylene, brassinolide, or the like) of appropriate concentration.

As the regeneration method from the transformed plant cell or the like to the transgenic plant, the following method is adopted, for example. Specifically, the transformed callus cell is transferred on media with hormones of different types and concentrations to form an adventitious embryo, and then a complete plant is obtained. Examples of the media used include a LS medium, a MS medium, and the like. The production steps of the transgenic plant in the present invention include: steps of introducing the plant expression vector in which the DNAs of the present invention are inserted into the host cell to obtain a transformed plant cell, and regenerating a transgenic plant from the transformed plant cell.

Moreover, the transgenic plant in which the DNAs of the present invention are introduced can be produced by crossing. Specifically, a plant which contains the DNAs (resistance genes) of the present invention is crossed with a plant which is crossable with the plant but does not contain the DNAs of the present invention. Thereby, the DNAs of the present invention are introduced into the plant which does not contain the DNAs of the present invention, and thus the resistance to the pathogens can be provided.

Once the transgenic plant having the chromosomes in which the DNAs of the present invention are introduced is obtained, a progeny can be obtained from the plant through sexual reproduction or asexual reproduction. In addition, seeds or the like are obtained from the plant, the progeny, or a clone thereof, and the plant can be produced in mass therefrom.

Moreover, a portion of the transgenic plant of the present invention can be used as, for example, foods and the like as agricultural crops. The present invention includes “a portion or a propagation material” of the transgenic plant of the present invention, such as a plant organ (for example, seed, leaf, petal, stem, root, or the like), a plant tissue (for example, epidermis, phloem, parenchyma, xylem, vascular bundle, or the like), or a plant cultured cell.

To obtain plant seeds from the transgenic plant, for example, the transgenic plant is taken out from the rooting medium, transplanted to a pot with a soil containing water, grown at a certain temperature, and caused to flower, and finally seeds are formed. Meanwhile, to produce the plant from the seed, for example, when the seeds formed on the transgenic plant mature, the seeds are isolated, seeded into a soil containing water, and grown at certain temperature and illumination to produce the plant. The plant thus nurtured is a diseases-stress tolerant plant that has acquired the resistance to multiple pathogens by the expression of the combination of the DNAs thus introduced.

### EXAMPLES

Hereinbelow, the present invention will be described in more details using Examples. However, the technical scope of the present invention is not limited to Examples below.

### Example 1

### Search for Resistance Genes by SSLP (Simple Sequence Length Polymorphism) Analysis

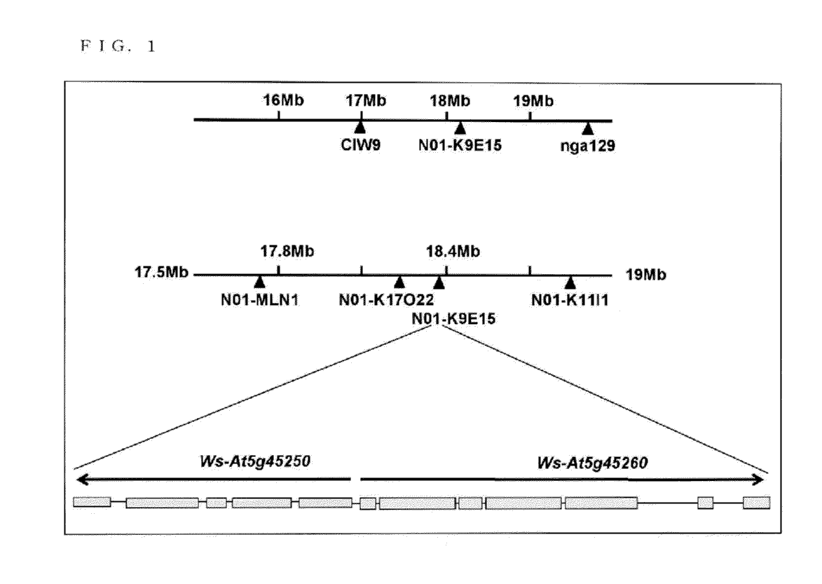

The Arabidopsis ecotype Wassilewskija (Ws-0) resistant to a pathogen of Cruciferous vegetable anthracnose (Colletotrichum higginsianum) was crossed with susceptible Columbia (Col-0), and F1 seeds were obtained. Furthermore, the F1 individuals were self-pollinated, and F2 seeds were obtained. The Arabidopsis F1 plants were grown (at 22° C. and cycles of a 12-hour light period and 12-hour dark period) for four weeks after seeded. Then, 5×10⁵/ml of an anthracnose pathogen spore suspension was sprayed on and inoculated into the plants. After six days, an assay was conducted, and all showed the resistance. Next, the F2 plants were grown similarly, and 5×10⁵/ml of an anthracnose pathogen spore suspension was sprayed on and inoculated into the plants. As a result, it was found out that the resistant individuals and the susceptible individuals were segregated in 3:1, that the resistance was dominant, and that this resistance was controlled at one locus or multiple loci adjacent to each other. Hence, it was expected that, when an individual having a susceptible phenotype to the pathogen was selected from the F2 group, the individual would have a homozygous Col-0 gene (alleles) at the corresponding target locus of Ws-0. Thus, it was also expected that, when the chromosome structures of a number of F2 individuals selected based on phenotype were examined by polymorphic markers, the alleles of the target genes existed in the vicinity of a region where the Col type was shown most frequently with the homozygous genotype. Based on the expectations, an SSLP analysis was conducted to identify the target genes. As a result, a region where the target genes existed was successfully narrowed down within a region between the polymorphic markers N01-K₁₁I1 and N01-K₁₇O22 (FIG. 1).

### Example 2

### Analysis of Relationship Between Resistance to Anthracnose Pathogen and Mutations in RPS4 Gene (At5g45250) and RCH2 Gene (At5g45260)

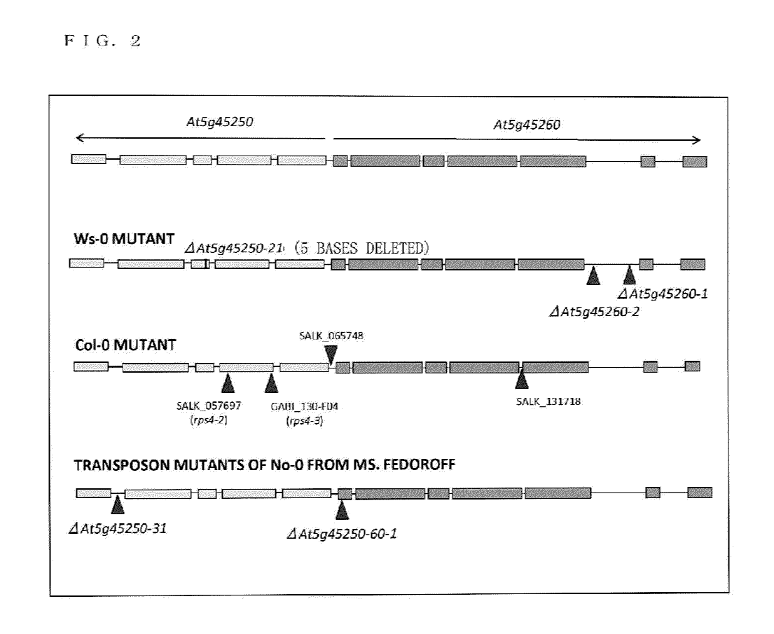

By the method of Example 1, it was no longer possible to generate polymorphic markers utilizable in narrowing down the region where the target genes existed, and the SSLP analysis came to the limit. For this reason, approximately 30,000 lines were screened using a T-DNA insertion library of the resistant ecotype Ws-0. As a result, obtained were eight individuals which were T-DNA inserted mutants susceptible to anthracnose. The base sequences of Ws-At5g45250 and Ws-At5g45260 of these mutants were analyzed, and mutants in which mutations were introduced into these genes were discovered. A mutant ΔAt5g45250-21 having five bases deleted from At5g45250 as well as mutants ΔAt5g45260-1 and ΔAt5g45260-2 having T-DNA tags inserted in At5g45260 showed susceptibility to the anthracnose pathogen (FIG. 2, “Ws-0 mutant”).

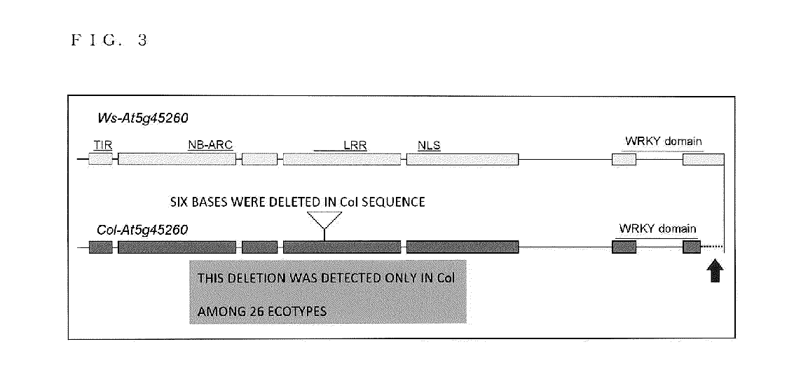

The present inventors had elucidated that Ws-At5g45250 was a resistance gene (Japanese Patent Application No. 2007-86343). It was also assumed that At5g45260 was possibly a resistance gene. For this reason, the At5g45260 amino acid sequences were compared between the ecotype Ws-0 and the ecotype Col-0 which were respectively resistant and susceptible to the anthracnose pathogen (FIG. 3). In Col-At5g45260, approximately 80 to 90 amino acids were deleted from the WRKY domain toward the C terminal. Similarly to Col-At5g45260, the ecotypes Cvi-0 and Bur-0 in which deletions toward the C terminal were recognized also showed susceptibility to the anthracnose pathogen. From this, it was found out that At5g45260 was also involved in the resistance to the anthracnose pathogen, and that the presence of at least approximately 80 to 90 amino acids from the WRKY domain of At5g45260 toward the C terminal was important to acquire the resistance.

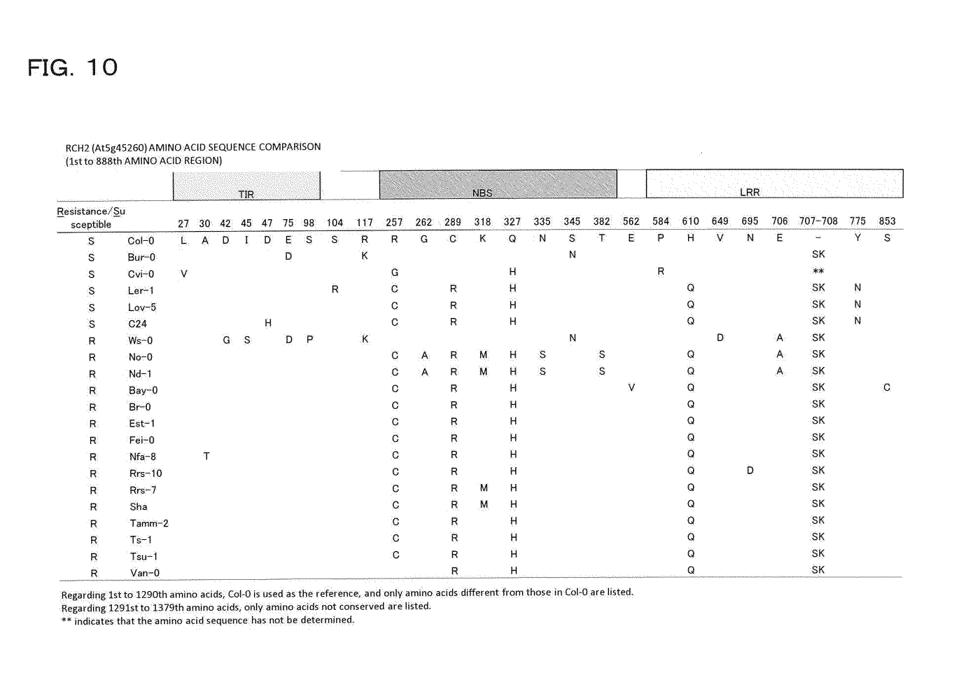

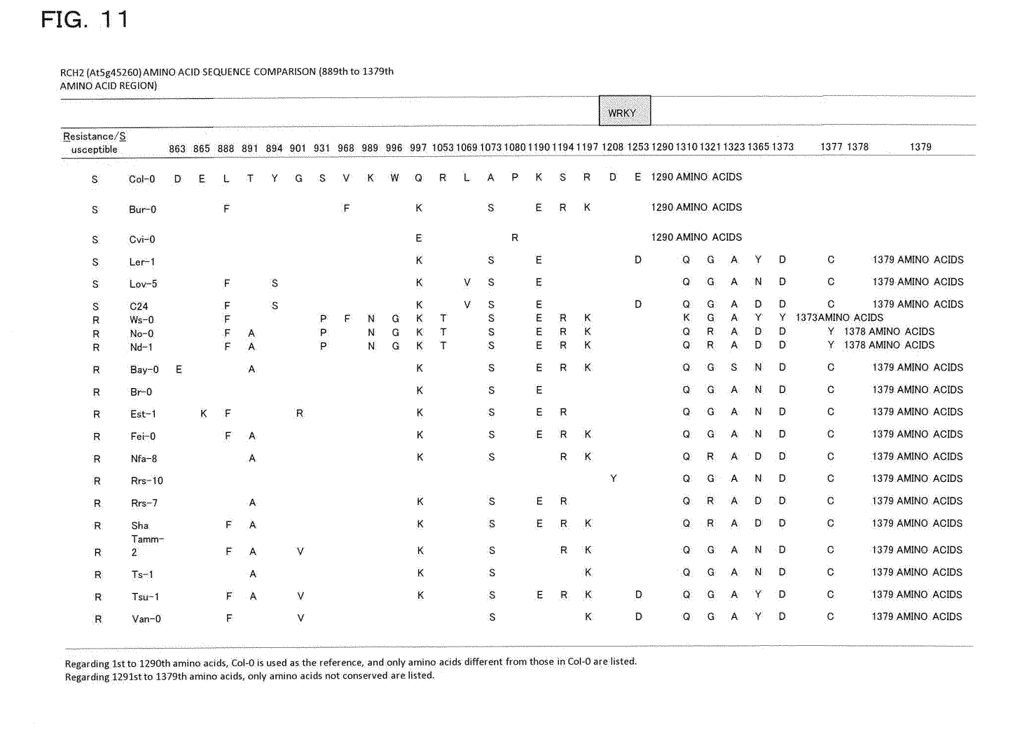

Furthermore, in the ecotypes (Ler-1, Lov-5, C24) susceptible to the anthracnose pathogen, the 775th tyrosine was substituted with asparagine. It was indicated that this amino acid was important in the resistance (FIGS. 10 and 11).

### Example 3

### Analysis of Relationship Between Resistance to Bacterial Leaf Spot having avrRps4 Gene and Mutations in At5g45250 and At5g45260

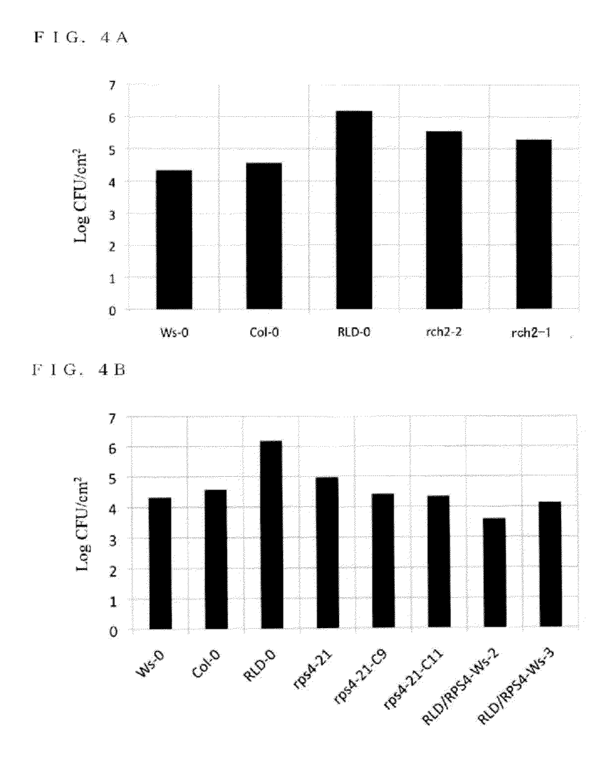

The resistance to a plant pathogen was examined using a bacterium of tomato leaf spot (Pseudomonas syringae pv. tomato) having the avrRps4 gene and known to infect Arabidopsis also. The bacterium was shake-cultured overnight in a King's B liquid medium to which kanamycin (25 μg/ml) and rifampicin (25 μg/ml) had been added. The bacterial suspension was prepared to 1×10⁵ (cfu)/ml. This bacterial suspension was injected into a rosette leaf of Arabidopsis grown (at 22° C. and cycles of an 8-hour light period and a 16-hour dark period) for seven weeks after seeded, in such a way that a 1-ml needless syringe was pressed on the back side of the leaf, and a pressure was applied thereon. Thereby, the pathogen was inoculated. On the third day after the inoculation, the inoculated leaf was cut out with a cork borer and fragmented in a 10-mM MgSO₄ liquid. The bacterial suspension was seeded on a solid medium. Two days later, the number of colonies appeared was counted. Thereby, the growth of the bacterium per leaf area was determined. FIGS. 4A and 4B show the results of the susceptibility (growth of the pathogenic bacterium) of plants (wild strains, overexpressors, gene disruptants) inoculated with the bacterium of tomato leaf spot.

(1) Relationship Between At5g45260 and Susceptibility to Bacterial Leaf Spot (FIG. 4A)

In the mutants having At5g45260 disrupted, the growth of the bacterium was high relative to that in the Ws-0 wildtype, and the susceptibility to bacterial leaf spot was increased.

(2) Relationship Between At5g45250 and Susceptibility to Bacterial Leaf Spot (FIG. 4B)

In the mutants having At5g45250 disrupted, the growth of the bacteria was also high relative to that in the Ws-0 wildtype, and the susceptibility to bacterial leaf spot was increased. Additionally, revertants which were each obtained by transforming the At5g45250-disrupted mutant with approximately 6 kb of the Ws-At5g45250 gene including approximately 2 kb of a promoter region showed similar resistance to that of the Ws-0 wildtype. Moreover, transformants which were each obtained by introducing approximately 6 kb of the Ws-At5g45250 gene including approximately 2 kb of a promoter region into an ecotype RLD susceptible to the anthracnose pathogen and bacterial leaf spot showed strong resistance to bacterial leaf spot.

From the above, it was found out that both At5g45250 and At5g45260 were essential for expression of resistance to bacterial leaf spot. So far, it had been reported that only At5g45250 was involved in the expression of the resistance to bacterial leaf spot (Non Patent Literature 2). However, from this result, it was shown that At5g45260 was also involved therein. Particularly, RLD-0, which was an only susceptible ecotype among the reported Arabidopsis ecotypes, acquired the resistance to the bacterial leaf spot and anthracnose pathogens by introducing Ws-At5g45250 into RLD-0. Therefore, it was clarified that RLD-0 originally had At5g45260 for resistance to these pathogens, and that the cause of the susceptibility was At5g45250.

### Example 4

### Analysis of Relationship Between Mutations in At5g45250, At5g45260 of Arabidopsis Ecotypes No-0, Ws-0 and Resistance to Anthracnose Pathogen, Bacterial Wilt Pathogen

First, No-0 from Ms. Fedoroff, which was reported to be resistant to a bacterial wilt pathogen (Non Patent Literature 12), was examined on the resistance to the anthracnose pathogen and bacterial leaf spot. As a result, this ecotype showed the resistance to both of the pathogens. Then, using transposon mutants of No-0, the relationship between mutations in At5g45250, At5g45260 and the resistance to these pathogens was examined (FIG. 2 “transposon mutants of No-0 from Ms. Fedoroff”). As a result, a mutant ΔAt5g45250-31 (rps4-31) having a transposon tag inserted into At5g45250 showed susceptibility to the anthracnose pathogen. Additionally, a mutant ΔAt5g45250-60-1 having transposon tags inserted into a promoter region of At5g45250 and into At5g45260 also showed susceptibility to the anthracnose pathogen. In the Arabidopsis ecotype No-0 (from Ms. Fedoroff) also, At5g45250 and At5g45260 were involved in the resistance to the anthracnose pathogen.

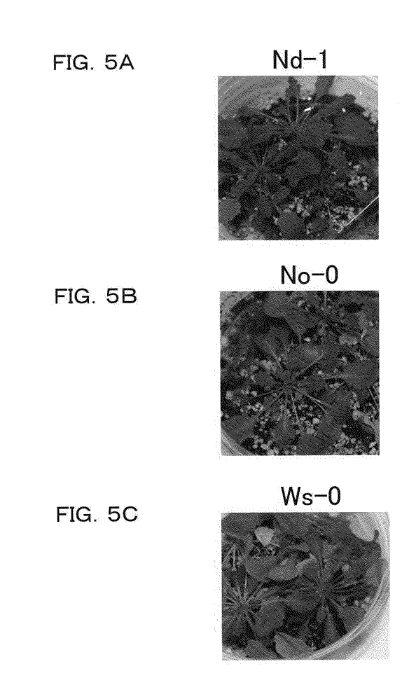

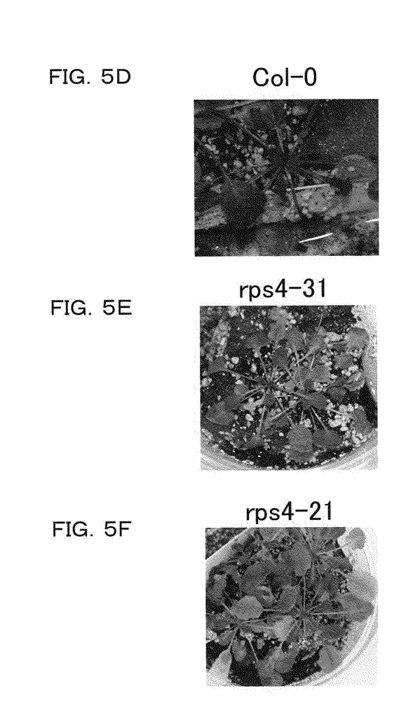

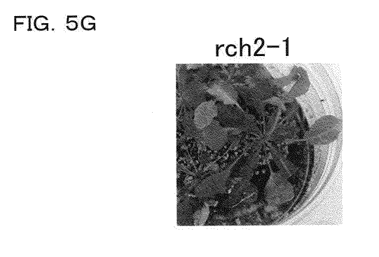

A bacterial wilt pathogen suspension was prepared to 1×10⁸ (cfu)/ml. Arabidopsis was grown (at 22° C. and cycles of an 8-hour light period and a 16-hour dark period) on rock wool for seven weeks after seeded. The root was cut off together with the rock wool, and 20 ml or more of the bacterial suspension was poured onto the root portion thus cut. The disease onset was assayed on the seventh day after the inoculation. FIGS. 5A to 5G show the results of the susceptibility of plants (wild strains, gene disruptants) inoculated with the bacterial wilt pathogen.

The ecotypes Nd-1, No-0, and Ws-0 were resistant to bacterial wilt (FIGS. 5A to 5C), whereas Col-0 was susceptible (FIG. 5D). It was reported that ΔAt5g45250-60-1 was susceptible to the bacterial wilt pathogen (Non Patent Literature 12). The present invention proved that the mutant ΔAt5g45250-31 (rps4-31) of No-0 from Ms. Fedoroff having the tag inserted into At5g45250 was susceptible to the bacterial wilt pathogen (FIG. 5E). A mutant ΔAt5g45250-21 (rps4-21) of the ecotype Ws-0 having five bases deleted in At5g45250 and a mutant ΔAt5g45260-1 (rch2-1) of Ws-0 having a T-DNA tag inserted in At5g45260 showed strong susceptibility to the bacterial wilt pathogen (FIGS. 5F and 5G). The above results suggest that, if a plant susceptible to these pathogens is transformed with At5g45250 and At5g45260 of Ws-0 and No-0 from Ms. Fedoroff, a plant resistant to the anthracnose pathogen, the bacterial leaf spot and the bacterial wilt pathogen can be produced.

### Example 5

### Introduction of Resistance Genes into Arabidopsis Ecotypes Col-0, RLD-0 Susceptible to Anthracnose Pathogen, and Effects of Introduction

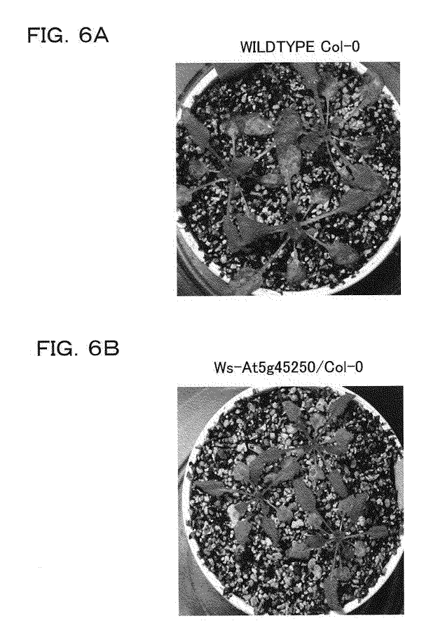

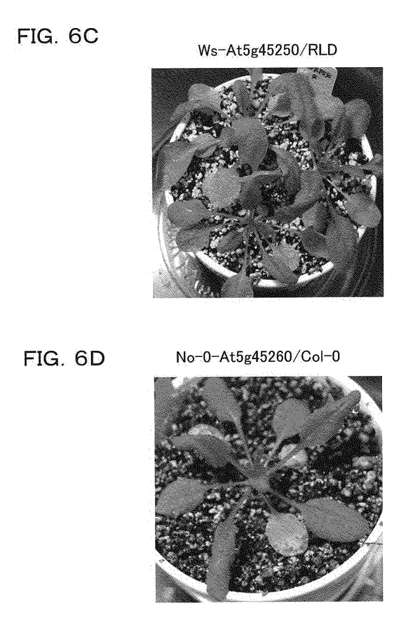

Into the Arabidopsis ecotypes Col-0 and RLD-0 susceptible to the anthracnose pathogen, At5g45250 or At5g45260 of the ecotype Ws-0 resistant to the anthracnose pathogen was introduced, and it was examined whether or not the ecotypes acquired the resistance to the anthracnose pathogen (FIGS. 6A to 6D). As a result, transformants obtained by introducing the resistance gene Ws-At5g45250 into the wildtype Col-0 showed the resistance to this fungal pathogen, but the plants were dwarfed (FIG. 6B). Meanwhile, transformants obtained by introducing the resistance gene Ws-At5g45250 into the wildtype RLD normally grew, and showed the resistance to this fungal pathogen (FIG. 6C). Transformants obtained by introducing the resistance gene No-0-At5g45260 from Ms. Fedoroff into the wildtype Col-0 normally grew, and showed the resistance to fungal pathogen (FIG. 6D). From the above, it was found out that, even if anthracnose pathogen-resistant Ws-At5g45250 was introduced into the wildtype Col-0, the resultant plants were not able to be stably maintained, but that the transformants obtained by introducing anthracnose pathogen-resistant No-0-At5g45260 from Ms. Fedoroff were stably maintained and were resistant to the anthracnose pathogen. RLD-0 originally has resistant At5g45260, and, when Resistant Ws-At5g45250 was introduced thereinto, RLD-0 was stably maintained in the plant, which showed the resistance to the anthracnose pathogen. By introducing two different resistance genes in this manner, the plants showing resistance to the anthracnose pathogen were successfully produced.

Note that Col-0 having No-0-At5g45260 from Ms. Fedoroff introduced thereinto has At5g45250 for resistance to bacterial leaf spot and At5g45260 for resistance to bacterial wilt. Accordingly, it would be easily understood by those skilled in the art that such Col-0 shows resistance to both bacterial leaf spot and bacterial wilt.

### Example 6

### Relationship Between Susceptibility of Various Arabidopsis Ecotypes to Anthracnose and Amino Acid Sequences of At5g45250, At5g45260

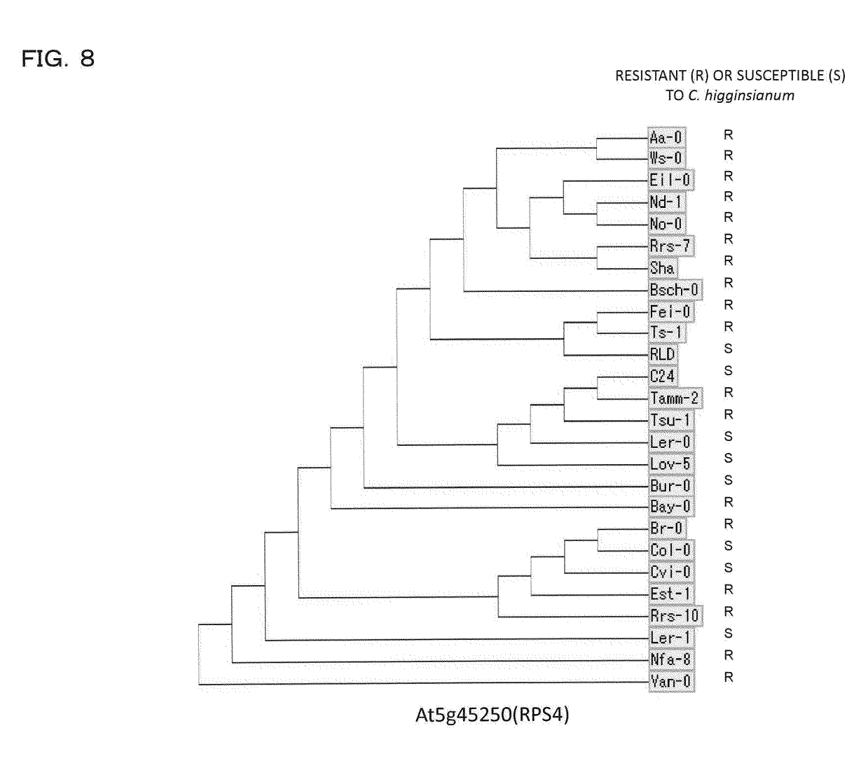

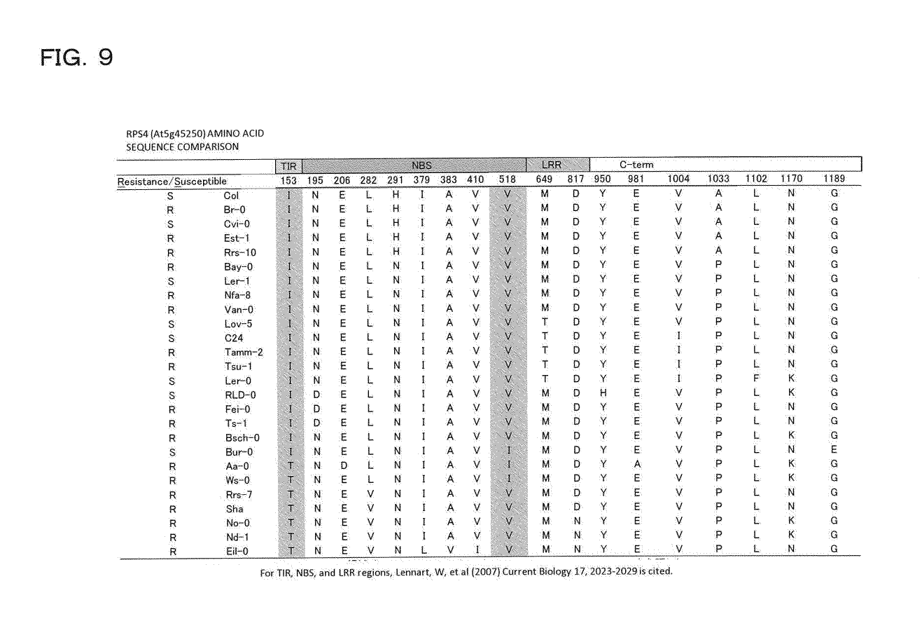

The Arabidopsis ecotypes were examined on the susceptibility to anthracnose (FIGS. 7 and 8). A phylogenetic tree analysis was conducted on the amino acid sequences of At5g45250 and At5g45260 by utilizing SNP analysis data on these ecotypes of Arabidopsis (Non Patent Literature 10) (FIGS. 7 and 8). As to At5g45250, the ecotypes which were susceptible and resistance to anthracnose were present randomly when classified. The At5g45250 amino acid sequences of the ecotypes were compared with one another. The 950th tyrosine was substituted with histidine in RLD-0 susceptible to all of Cruciferous vegetable anthracnose, bacterial leaf spot, and bacterial wilt (FIG. 9). Hence, a construct was created in which the 950th tyrosine of Ws-At5g45250 in Ws-0 resistance to anthracnose was substituted with histidine. The construct was introduced into a disruptant ΔAt5g45250-21 (rps4-21) of Ws-At5g45250 using Ws-0 as a background. This transformant showed susceptibility to anthracnose, and was not able to recover the resistance. Thus, it was indicated that this amino acid was important in the resistance. Meanwhile, as a result of the phylogenetic tree analysis on the At5g45260 amino acid sequence, as to At5g45260, susceptible ecotypes were classified into one group (FIG. 7). In this group (Ler-1, Lov-5, C24), the 775th tyrosine was substituted with asparagine as described in Example 2. It was indicated that this amino acid was important in the resistance (FIGS. 10 and 11).

### Example 7

### Introduction of At5g45250 and At5g45260 Derived from Arabidopsis Ecotype Ws-0 into Ecotype Col-0 Susceptible to Anthracnose Pathogen, and Effects of Introduction

The results in Examples 2 to 6 indicated that the two genes At5g45250 and At5g45260 were essential for resistance of plant to the anthracnose pathogen, and that, by introducing these two genes into an ecotype plant susceptible to the anthracnose pathogen, the plant was successfully transformed to an ecotype resistant to the anthracnose pathogen. To reconfirm this fact, the present inventors introduced At5g45250 and At5g45260 of the Arabidopsis ecotype Ws-0 resistant to the anthracnose pathogen into the ecotype Col-0 susceptible to the anthracnose pathogen, and it was examined whether or not the ecotype acquired the resistance to the anthracnose pathogen.

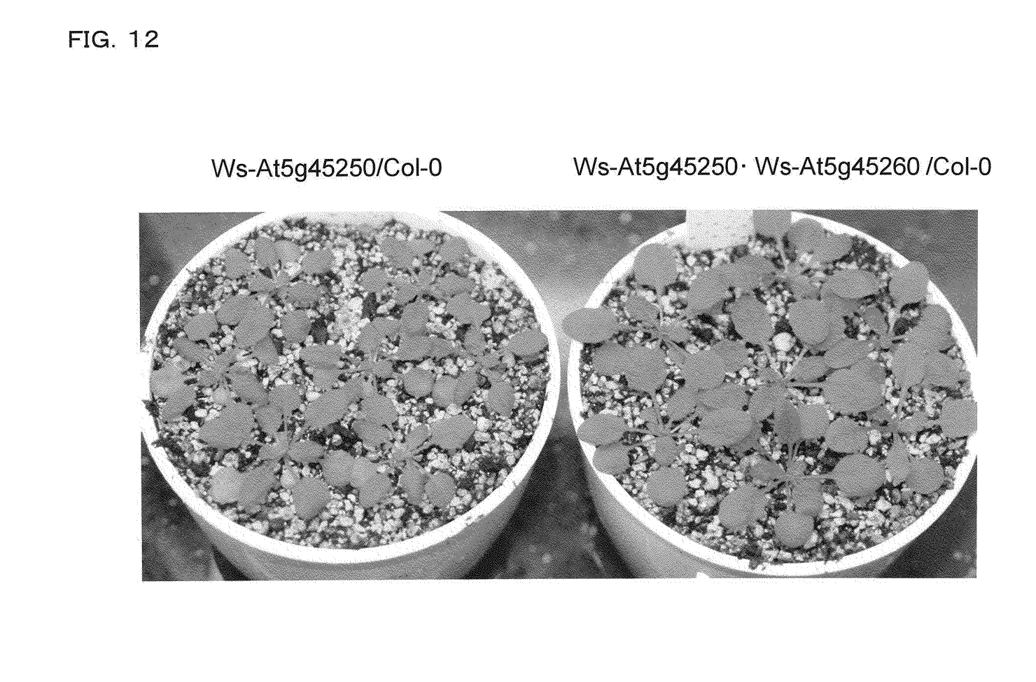

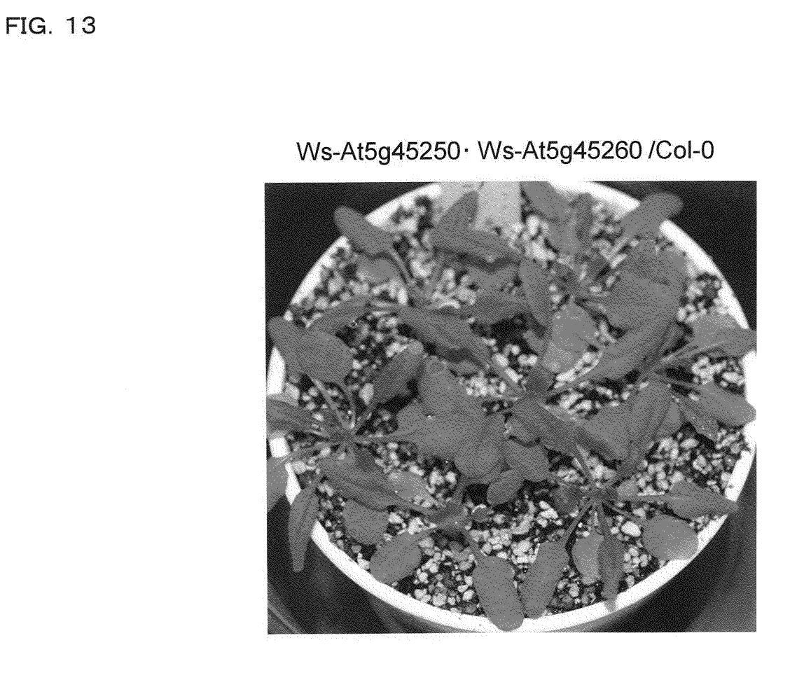

As a result, as already proved in Example 5 (FIG. 6B), transformants obtained by introducing the resistance gene Ws-At5g45250 into the wildtype Col-0 showed the resistance to this fugal pathogen, but the plants were dwarfed (FIG. 12, left). Transformants obtained by introducing Ws-At5g45260 derived from the ecotype Ws-0 resistant to the anthracnose pathogen into the dwarfed transformants were stably maintained in the plants, normally grew without being dwarfed (FIG. 12, right), and acquired the resistance to the anthracnose pathogen (FIG. 13). From the above, it was reconfirmed that, by introducing two different resistance genes, a plant showing the resistance to the anthracnose pathogen was successfully produced. Meanwhile, instances are reported that, when a disease resistance gene alone is introduced, the plant is dwarfed generally. Nevertheless, it was also reconfirmed that such a problem of a dwarfed plant in producing resistant plants were successfully avoided by introducing multiple genes in combination.

Note that Col-0 having Ws-At5g45250 and Ws-At5g45260 derived from Ws-0 introduced at the same time has Ws-At5g45250 for resistance to bacterial leaf spot and Ws-At5g45260 for resistance to bacterial wilt. Accordingly, it would be easily understood by those skilled in the art that such Col-0 shows resistance to both bacterial leaf spot and bacterial wilt.

### Example 8

### Introduction of At5g45250 and At5g45260 Derived from Arabidopsis Ecotype Ws-0 into Japanese Mustard Spinach Cultivar “Osome” Susceptible to Anthracnose Pathogen, and Effects of Introduction

The results in Examples 2 to 6 indicated that the two genes At5g45250 and At5g45260 were essential for resistance of plant to the anthracnose pathogen, and that, by introducing these two genes into an ecotype plant susceptible to the anthracnose pathogen, the plant was successfully transformed to an ecotype resistant to the anthracnose pathogen. To reconfirm this fact, the present inventors introduced At5g45250 and At5g45260 of the Arabidopsis ecotype Ws-0 resistant to the anthracnose pathogen into the Japanese mustard spinach cultivar “Osome” susceptible to the anthracnose pathogen, and it was examined whether or not the cultivar acquired the resistance to the anthracnose pathogen.

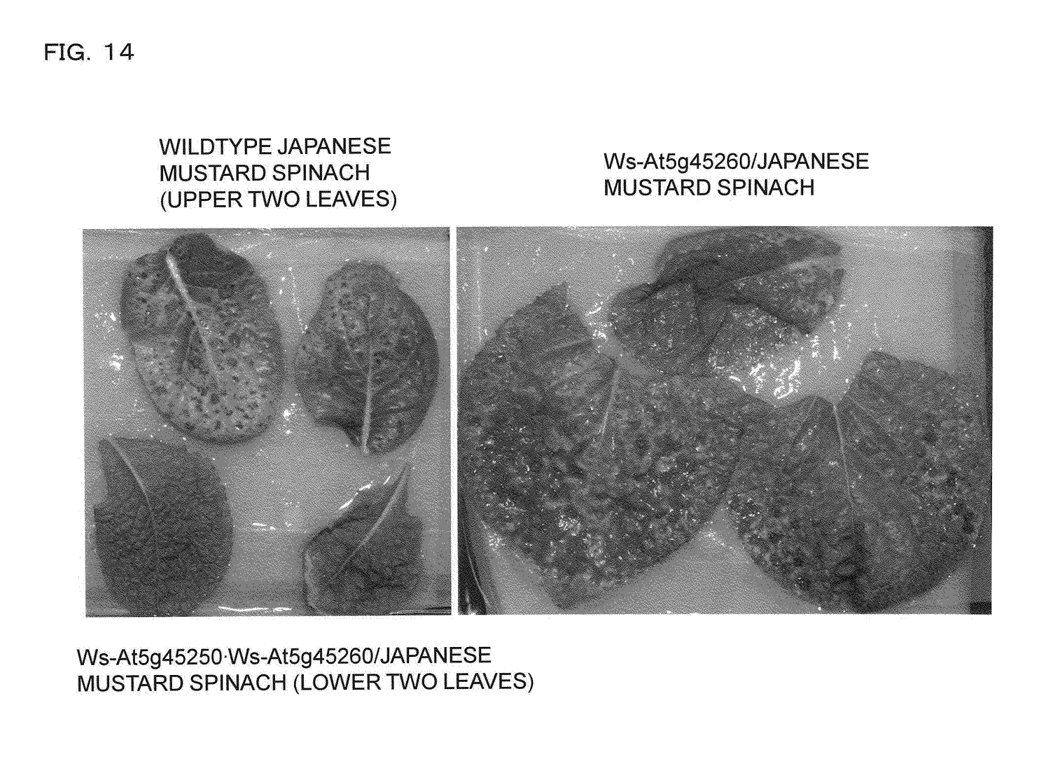

As a result, most of transformants obtained by introducing Ws-At5g45250 into the wildtype Japanese mustard spinach were dwarfed, and the individuals were not able to be maintained. Individual plants that somehow survived were dwarfed. Moreover, transformants obtained by introducing the resistance gene Ws-At5g45260 into the wildtype Japanese mustard spinach were not able to stably acquire the resistance to the anthracnose pathogen (FIG. 14, right). Meanwhile, transformants obtained by introducing At5g45250 and At5g45260 of the Arabidopsis ecotype Ws-0 into the wildtype Japanese mustard spinach at the same time were stably maintained in the plants, and the plants also normally grew and were resistant to the anthracnose pathogen (FIG. 14, left). From the above, it was reconfirmed that, by introducing two different resistance genes, a plant showing the resistance to the anthracnose pathogen was successfully produced. Meanwhile, instances are reported that, when a disease resistance gene alone is introduced, the plant is dwarfed generally. Nevertheless, it was also reconfirmed that such a problem of a dwarfed plant in producing resistant plants were successfully avoided by introducing multiple genes in combination.

Note that the Japanese mustard spinach having Ws-At5g45250 and Ws-At5g45260 derived from Ws-0 introduced at the same time has Ws-At5g45250 for resistance to bacterial leaf spot and Ws-At5g45260 for resistance to bacterial wilt. Accordingly, it would be easily understood by those skilled in the art that such Japanese mustard spinach shows resistance to both bacterial leaf spot and bacterial wilt.

### INDUSTRIAL APPLICABILITY

The present invention is suitably utilizable in the fields of breeding, for example, Cruciferous crops such as Chinese cabbage, Japanese mustard spinach, turnip, cabbage, rapeseed, and white radish and Solanaceae plants such as tomato, eggplant, bell pepper, and potato. The use of the present invention makes it possible to protect these crops from diseases due to pathogens of Cruciferous vegetable anthracnose, bacterial leaf spot, and bacterial wilt, and to improve the productivity of these crops.

1. A method for providing a plant with resistance to two or more pathogens, the method characterized by comprising:
introducing a combination of two or more genes into the plant, wherein
the genes do not provide the plant with the resistance to the two or more pathogens when each of the genes is introduced alone, but provide the plant with the resistance to the pathogens when introduced in the combination.

2. The method according to claim 1, wherein
the pathogens are pathogens of Cruciferous vegetable anthracnose and bacterial leaf spot of tomato, and
the combination of the genes is a combination of an RPS4 gene and an RCH2 gene.

3. The method according to claim 2, wherein
the RPS4 gene and the RCH2 gene are derived from an Arabidopsis ecotype selected from the group consisting of Ws-0, No-0, Nd-1, Aa-0, Eil-0, Rrs-7, Sha, Tamm-2, Tsu-1, Fei-0, Ts-1, Bsch-0, Br-0, Est-1, Rrs-10, Van-0, Nfa-8, and Bay-0.

4. The method according to claim 2, wherein the pathogens further include a bacterial wilt pathogen.

5. A transgenic plant provided with resistance to two or more pathogens, comprising:
a combination of two or more genes introduced therein, wherein
the genes do not provide the plant with the resistance to the two or more pathogens when each of the genes is introduced alone, but provide the plant with the resistance to the pathogens when introduced in the combination.

6. The transgenic plant according to claim 5, wherein
the pathogens are pathogens of Cruciferous vegetable anthracnose and bacterial leaf spot of tomato, and
the combination of the genes is a combination of an RPS4 gene and an RCH2 gene.

7. The transgenic plant according to claim 6, wherein
the RPS4 gene and the RCH2 gene are derived from an Arabidopsis ecotype selected from the group consisting of Ws-0, No-0, Nd-1, Aa-0, Eil-0, Rrs-7, Sha, Tamm-2, Tsu-1, Fei-0, Ts-1, Bsch-0, Br-0, Est-1, Rrs-10, Van-0, Nfa-8, and Bay-0.

8. The transgenic plant according to claim 6, which further has resistance to a bacterial wilt pathogen.

9. A transgenic plant which is any one of a progeny and a clone of the transgenic plant according to claim 5.

10. Any one of a portion and a propagation material of the transgenic plant according to claim 5.


SEQUENCE LISTING (12 sequences)

SEQ ID NO: 1 - DNA, 3654 bp, Arabidopsis thaliana Ws-0
  CDS (1)..(3651)
atggagacatcatctatttccactgtggaagacaagccaccgcagcatcaggtgttcatcaatttccgtggggcagatttgcgccggagattcgtcagccatctcgtaacggccttgaaattgaacaacatcaacgtctttatcgacgactatgaagacagaggtcaacctctagatgtactgctgaagaggatagaggagtccaaaatcgttttggctatcttctccggcaactacaccgagtcagtctggtgcgtgagagagctcgagaagatcaaggattgtacggatgaaggaacactcgttgcgattccaatcttctacaagctggagccatccaccgttagagatttgaaaggaaagttcggtgatagatttaggagtatggctaagggtgatgagaggaaaaaaaaatggaaggaagcttttaacttgattcctaacattatgggcatcaccattgacaagaaaagtgtggagagtgagaaagtcaatgaaattgtgaaggcggtaaagacagcgttgaccggaataccaccggagggaagtcacaatgccgtcgtgggtgctttaggtaatagcaacgctggaacttcctcaggagacaaaaagcatgagacttttggaaacgaacaacggttaaaagatttggaagagaagttggatcgtgataaatacaagggaactcgtatcattggagttgttgggatgcccggaattggtaaaaccacactcctgaaagagctatataagacgtggcagggcaagttttcgaggcatgcgctaatcgatcaaatccgtgtaaagtccaagcacttggagttggatcgcttgcctcaaatgctcttaggcgagttgtcgaagttaaacaatcctcacgtagacaatctcaaagatccatacagtcaactgcatgaacgcaaagtgcttgttgtacttgatgatgttagtaaaagggaacaaatagatgctcttcgtgagatactagactggattaaggagggtaaggagggaagcagggttgtcattgcaacaagcgacatgtccttaacaaatggtttggttgatgatacttacatggttcaaaatttgaaccacagagatagcttacaactatttcactatcatgcctttattgatgatcaagccaatcctcaaaagaaagatttcatgaagctgtcagaagggtttgtacattacgccagaggccatccactagccctcaaagtattgggtggagagcttaataagaaaagtatggatcattggaattcgaaaatgaagaaacttgcacagagccccagccctaatattgtcagtgtcttccaagtgagttatgatgaattgactacagcgcagaaagatgcatttctcgacatagcctgtttcagatcacaggacaaggattatgtagaaagtctactggcttcatctgaccttggttcggctgaagcaatgagtgcagtcaaatctctcacggataagttcctgattaatacttgtgatggccgagtggagatgcatgatctattatataaattttcaagggaaattgatctgaaggcatctaatcaggatggtagcagacaacggaggctgtggctccatcaacacataatcaagggaggcataatcaatgtattgcaaaataaaatgaaagctgccaatgttagaggtattttcctagacttgtctgaagtggaagacgaaacgagcttagaccgcgaccacttcataaatatgggaaatctccggtatctcaagttctacaattcccattgtccacaggaatgtaaaaccaacaataaaatcaacatccctgataaacttaagctaccattgaaagaagttcgatgcctccactggctgaaattcccattggagacacttccaaacgatttcaacccaattaatcttgtcgaccttaagctgccctacagtgaaatggaacaactttgggagggtgacaaggacacaccatgcttaaggtgggtcgatctcaatcactcaagtaagttgtgcagcttgtcagggttatcaaaagctgaaaagcttcaaaggttgaaccttgaaggttgcacaacactgaaagcgtttccacatgatatgaaaaaaatgaaaatgcttgctttcttgaatctgaaggggtgtacaagtctcgaatctcttccagagatgaatttgatttctctgaaaacacttactctcagcggctgctcaacttttaaggaatttccgttgatttcagataatatagaaactctatacttagatggcacagcaataagtcagcttcctatgaacatggagaagcttcagagacttgttgtattgaatatgaaagactgcaaaatgctagaggaaattccaggccgtgttggtgagctgaaagctcttcaagaactgatactctctgattgtttaaatctcaagattttcccagaaatcgacattagctttttaaacattttacttttggatgggacagccattgaagtgatgccacaattaccctcagtgcagtatttgtgcttaagcagaaatgctaagatcagctgccttcctgttggaatcagtcagctttctcaacttaaatggctggacctgaagtattgtacgagtcttacatcagttccagagtttccaccaaatcttcagtgcttagatgcacacggctgtagttcgctgaagacagtttcaaagcctttggcccgtatcatgccaactgaacagaatcattccacattcattttcaccaactgtgagaacctggaacaagctgcaaaggaggaaatcacatcgtatgctcaaaggaaatgccagctgttatcatatgctcggaaacgctacaatgggggtcttgtttcagaatctttgttcagcacttgctttcctggatgtgaagtgccttcttggttttgtcatgaaacagttggatctgagttagaagtaaaactcctcccacattggcatgacaagaagcttgccgggattgctctatgtgctgtagtctcatgtcttgacccccaagatcaagtcagccgcttgtcagtgacctgcacctttaaagtaaaagatgaagacaagtcttgggtcccatatacttgtccagtaggaagttggaccagacatggaggcgggaaagataagattgagctagaccatgtcttcattggatacaccagttgcccacatactataaaatgtcatgaagaaggtaactcagatgaatgcaatcctaccgaagcctctcttaaatttactgtgacaggaggtacaagtgagaatggaaaatacaaggtgttgaagtgtggtttaagtttggtgtatgctaaggataaagacaaaaatagtgctcttgaaacaaagtacgatatgcttattggaaagagctttcaagaaacttcagaaggggttgatggaagggtaaagaaaacgaaaggaaagtacgttatgcctgttgaaaagaactttcaagaaactacagaaggggttgatggaagagtaaagaaaaagaaaaaaacaagaatggataatggacggccaaagaagaagcagagatcaggaagagatgataatcaaactaggatgcaagtggaactccaagaaggaaatataaattcagtgatcatgcacacggttaagaatttctga

SEQ ID NO: 2 - protein, 1217 aa, Arabidopsis thaliana Ws-0
METSSISTVEDKPPQHQVFINFRGADLRRRFVSHLVTALKLNNINVFIDDYEDRGQPLDVLLKRIEESKIVLAIFSGNYTESVWCVRELEKIKDCTDEGTLVAIPIFYKLEPSTVRDLKGKFGDRFRSMAKGDERKKKWKEAFNLIPNIMGITIDKKSVESEKVNEIVKAVKTALTGIPPEGSHNAVVGALGNSNAGTSSGDKKHETFGNEQRLKDLEEKLDRDKYKGTRIIGVVGMPGIGKTTLLKELYKTWQGKFSRHALIDQIRVKSKHLELDRLPQMLLGELSKLNNPHVDNLKDPYSQLHERKVLVVLDDVSKREQIDALREILDWIKEGKEGSRVVIATSDMSLTNGLVDDTYMVQNLNHRDSLQLFHYHAFIDDQANPQKKDFMKLSEGFVHYARGHPLALKVLGGELNKKSMDHWNSKMKKLAQSPSPNIVSVFQVSYDELTTAQKDAFLDIACFRSQDKDYVESLLASSDLGSAEAMSAVKSLTDKFLINTCDGRVEMHDLLYKFSREIDLKASNQDGSRQRRLWLHQHIIKGGIINVLQNKMKAANVRGIFLDLSEVEDETSLDRDHFINMGNLRYLKFYNSHCPQECKTNNKINIPDKLKLPLKEVRCLHWLKFPLETLPNDFNPINLVDLKLPYSEMEQLWEGDKDTPCLRWVDLNHSSKLCSLSGLSKAEKLQRLNLEGCTTLKAFPHDMKKMKMLAFLNLKGCTSLESLPEMNLISLKTLTLSGCSTFKEFPLISDNIETLYLDGTAISQLPMNMEKLQRLVVLNMKDCKMLEEIPGRVGELKALQELILSDCLNLKIFPEIDISFLNILLLDGTAIEVMPQLPSVQYLCLSRNAKISCLPVGISQLSQLKWLDLKYCTSLTSVPEFPPNLQCLDAHGCSSLKTVSKPLARIMPTEQNHSTFIFTNCENLEQAAKEEITSYAQRKCQLLSYARKRYNGGLVSESLFSTCFPGCEVPSWFCHETVGSELEVKLLPHWHDKKLAGIALCAVVSCLDPQDQVSRLSVTCTFKVKDEDKSWVPYTCPVGSWTRHGGGKDKIELDHVFIGYTSCPHTIKCHEEGNSDECNPTEASLKFTVTGGTSENGKYKVLKCGLSLVYAKDKDKNSALETKYDMLIGKSFQETSEGVDGRVKKTKGKYVMPVEKNFQETTEGVDGRVKKKKKTRMDNGRPKKKQRSGRDDNQTRMQVELQEGNINSVIMHTVKNF

SEQ ID NO: 3 - DNA, 4122 bp, Arabidopsis thaliana Ws-0
  CDS (1)..(4119)
atgaccaattgtgaaaaggatgaggaattcgtgtgcatcagctgcgtagaagaggtacggtactctttcgtgagccacctctccgaagctctccgccgaaaaggcataaataatgtggtcgtaggtgtagatagcgatgatctgcttttcaaggagtctcaggcaaagatcgagaaagctggggtttctgtgatggttttacccggaaactgtgatccttccgatgtatggcttgacaagttcgccaaggttctcgagtgccaaaggaacaacaaggaccaggcggtggttccagtattgtacggtgacagtctattacgggaccaatggcttagcgagctggatttcaaaggcttatcacgaattcaccaatccaggaaggaatgtagtgactctatacttgtagaagagattgtgagagatgtgtacgagacgcacttttatgttggacgaattggaatctattcgaagctgctggagattgaaaacatggttaacaagcaaccgataggcatccgttgtgttggaatttggggtatgcctggcataggaaagacaacacttgctaaagcagtctttgaccaaatgtctagcgcctttgatgcttcttgttttatcgaagactatgacaaatcaattcatgagaagggtctttattgtttgctggaggaacaacttttgccgggcaatgatgcaaccattatgaaactgagctcgctcagagacagattgaacagtaagagagttcttgttgttctcgatgacgtgcgcaatgctctggttggggagtcctttctcgaggggtttgactggctaggacccggaagcctgatcatcataacctctagagataaacaagtgttttgcctttgcggaatcaatcaaatatatgaggtccagggtttaaatgagaaagaggctcgtcaacttttcttgctgtctgcgtctataaaggaggatatgggagagcagaatctccaggagttgtcagtgagagtaataaattatgctaatggaaacccgttagctatcaatgtttatggaagagagctgaaaggtaagaaaaaactctcagaaatggagactgcattcctcaaactcaagcgacgtcctccatttaagattgtcgatgcatttaaaagcacctatgacacactcagtgacaacgaaaagaacatttttttggacatagcttgtttcttccagggagaaaatgtcaactacgtgatacaactgcttgagggttgtggtttctttccacatgttgaaattgatgtccttgttgacaagtgtctggtaactatttcagaaaaccgagtttggttgcataagctgacccaggatatcggccgagaaatcataaatggagaaacagtacagatcgagaggcgcagaagactgtgggaaccttggagcatcaaatatttattagaatataatgaacacaaagcaaatggagaacctaaaacaaccttcaaacgtgctcagggctctgaagagatcgaaggcctgtttctagacacatcaaacttaagatttgatctgcagccctctgcctttaagaatatgttgaaccttagattgctcaaaatttattgttccaatcctgaagtccatcctgtaatcaatttcccaacaggctctctgcattctcttcctaatgagctaagactcctccattgggagaactatcctctgaaatctttgcctcagaattttgatcctaggcaccttgtcgaaatcaacatgccgtatagtcaacttcagaaactttggggtggaaccaagaacctggagatgttgaggacgatcaggctttgccattcccaccatctagttgatatcgatgatctcttaaaagctgaaaatcttgaggtaattgatctccaaggttgtacgagactgcagaatttcccagccgcaggtcgattgctacgtctacgagatgtaaatctctcaggttgcataaagattaaaagtgtcctagaaattccaccaaatattgagaaactacatctacagggaactggcatattagcattaccagtttccactgttaagccaaaccatagagagcttgtgaattttctaacagaaattccgggtctttcagaggcctcgaaacttgagcgtttaacaagtctgctggaatctaactcatcttgtcaagatcttgggaagcttatttgcttggagctgaaagattgctcttgtttgcagagtctgccaaacatggctaatttagatcttaatgttcttgatctctcgggttgctcaagtcttaattctattcagggtttccctcgttttctgaaacagttatatcttggtggcactgcaataagagaagtgccacaacttcctcaaagtctagaaatcttgaatgcacatggatcttgtttgcgaagtctgccaaacatggctaatttagaatttctcaaagttcttgatctctctggttgctcagagctcgagactattcagggttttcctcggaacctaaaagagttatattttgctggcactacgttaagagaagtgccccaacttcctttaagcctagaggtcttgaatgcacatggttctgactcggagaagcttcctatgcattacaagttcaacaattttttcgatctatctcaacaagtggtcaacgattttttcttgaaaacgctgacttatgtaaaacacataccaagagggtatacgcaggaactcatcaacaaagctccgactttcagcttcagtgcgccctcacatacaaatcaaaacgccacatttgatctgcaaccaggatcttctgtaatgacacgactaaatcattcatggaggaacacgcttgtgggatttggtatgctggtggaagttgcatttcccgaggactactgtgatgctacagattttggcataagttgtgtttgcagatggagcaacaaagaaggccgctcttgtaggatagaaagaaattttcattgttgggcaccagggaaagttgttccaaaagttcgaaaggatcatacgtttgtctttagtgatgtcaacatgcgcccaagtaccggtgaaggaaatgaccctgatatctgggctggattagttgtatttgagttctttcctatcaatcagcagacaaagtgtctaaatgataggttcacagtgacaagatgtggagtccgtgtaataaatgttgcaactggcaatacaagtcttgagaacatatcactagttttgtctttggatccagtggaggtttctggttatgaagtattgagagtcagctatgatgatttacaggagatggataaagttctatttctttacatagcgtctttgttcaatgacgaggatgttgattttgtggcaccacttattgccggtattgacttggatgttagctctgggctcaaggtcttagccgatgtgtctctcataagtgtatcatccaatggggaaatagtgatgcatagtttgcaaagacaaatgggcaaagaaatcctccatggacaatccatgctgctgtctgattgtgagagttccatgaccgagaatttgtctgacgtaccaaaaaaggagaagaaacatcgcgaaagtaaggtaaagaaagtggtttccataccggctatagacgagggagatctatggacttggcgaaagtacggtcaaaaagacatcttaggttctcgttttccaaggggttactacagatgcgcttacaagttcacgcatggttgtaaagctacaaaacaagtccaacggagcgagaccgattcaaacatgttagctattacttacctatctgagcataaccatccacggcccactaaacgcaaggctctcgctgactccactcgttccacttcctcctccatctgctcagccataactacctctgcctcatctagagtcttccaaaacaaagacgaaccaaataaaccccacttgccttcctcctccactcctcctggaaacgcggctgtcttgtttaaaatgacggacatggaggagtttcaggacaatatggaggtggataatgacgtcgtagatacacgtacactggcattgtttccagagtttcaacatcagccggaggaagaatacccttggtcaacattcttcgattattag

SEQ ID NO: 4 - protein, 1373 aa, Arabidopsis thaliana Ws-0
MTNCEKDEEFVCISCVEEVRYSFVSHLSEALRRKGINNVVVGVDSDDLLFKESQAKIEKAGVSVMVLPGNCDPSDVWLDKFAKVLECQRNNKDQAVVPVLYGDSLLRDQWLSELDFKGLSRIHQSRKECSDSILVEEIVRDVYETHFYVGRIGIYSKLLEIENMVNKQPIGIRCVGIWGMPGIGKTTLAKAVFDQMSSAFDASCFIEDYDKSIHEKGLYCLLEEQLLPGNDATIMKLSSLRDRLNSKRVLVVLDDVRNALVGESFLEGFDWLGPGSLIIITSRDKQVFCLCGINQIYEVQGLNEKEARQLFLLSASIKEDMGEQNLQELSVRVINYANGNPLAINVYGRELKGKKKLSEMETAFLKLKRRPPFKIVDAFKSTYDTLSDNEKNIFLDIACFFQGENVNYVIQLLEGCGFFPHVEIDVLVDKCLVTISENRVWLHKLTQDIGREIINGETVQIERRRRLWEPWSIKYLLEYNEHKANGEPKTTFKRAQGSEEIEGLFLDTSNLRFDLQPSAFKNMLNLRLLKIYCSNPEVHPVINFPTGSLHSLPNELRLLHWENYPLKSLPQNFDPRHLVEINMPYSQLQKLWGGTKNLEMLRTIRLCHSHHLVDIDDLLKAENLEVIDLQGCTRLQNFPAAGRLLRLRDVNLSGCIKIKSVLEIPPNIEKLHLQGTGILALPVSTVKPNHRELVNFLTEIPGLSEASKLERLTSLLESNSSCQDLGKLICLELKDCSCLQSLPNMANLDLNVLDLSGCSSLNSIQGFPRFLKQLYLGGTAIREVPQLPQSLEILNAHGSCLRSLPNMANLEFLKVLDLSGCSELETIQGFPRNLKELYFAGTTLREVPQLPLSLEVLNAHGSDSEKLPMHYKFNNFFDLSQQVVNDFFLKTLTYVKHIPRGYTQELINKAPTFSFSAPSHTNQNATFDLQPGSSVMTRLNHSWRNTLVGFGMLVEVAFPEDYCDATDFGISCVCRWSNKEGRSCRIERNFHCWAPGKVVPKVRKDHTFVFSDVNMRPSTGEGNDPDIWAGLVVFEFFPINQQTKCLNDRFTVTRCGVRVINVATGNTSLENISLVLSLDPVEVSGYEVLRVSYDDLQEMDKVLFLYIASLFNDEDVDFVAPLIAGIDLDVSSGLKVLADVSLISVSSNGEIVMHSLQRQMGKEILHGQSMLLSDCESSMTENLSDVPKKEKKHRESKVKKVVSIPAIDEGDLWTWRKYGQKDILGSRFPRGYYRCAYKFTHGCKATKQVQRSETDSNMLAITYLSEHNHPRPTKRKALADSTRSTSSSICSAITTSASSRVFQNKDEPNKPHLPSSSTPPGNAAVLFKMTDMEEFQDNMEVDNDVVDTRTLALFPEFQHQPEEEYPWSTFFDY

SEQ ID NO: 5 - DNA, 3654 bp, Arabidopsis thaliana No-0
  CDS (1)..(3651)
atggagacatcgtctatttccactgtggaagacaagccaccgcagcatcaggtgttcatcaatttccgtggggcagatttgcgccggagattcgtcagccatctcgtcacggccttgaaattgaacaacatcaacgtctttatcgacgactatgaagacagaggtcaacctctagatgtactgctgaagaggatagaggagtccaaaatcgttttggctatcttctccggcaactacaccgagtcagtctggtgcgtgagagagctggagaagatcaaagattgtacggatgaaggaacactcgttgcgattccaatcttctacaagctggagccatccaccgttagagatttgaaaggaaagttcggtgatagatttaggagtatggctaagggtgatgagaggaaaaaaaaatggaaggaagcttttaacttgattcctaacattatgggcatcaccattgacaagaaaagtgtggagagtgagaaagtcaatgaaattgtgaaggccgtgaagacagcgttgaccggaataccaccggagggaagtcacaatgccgtcgtgggtgctttaggtaatagcaacgctggaacttcctcaggagacaaaaagcatgagacttttggaaacgaacaacggttaaaagatttggaagagaagttggatcgtgataaatacaagggaactcgtatcattggagttgtcgggatgcccggaattggtaaaaccacactcctgaaagagctatataagacgtggcagggcaagttttcgaggcatgcgctaatcgatcaaatccgtgtaaagtccaagcacttggagttggatcgcttgcctcaaatggtcttaggcgagttatcgaagttaaacaatcctcacgtagacaatctcaaagatccatacagtcaactgcatgaacgcaaagtgcttgttgtacttgatgatgttagtaaaagggaacaaatagatgctcttcgtgagatactagactggattaaggagggtaaggagggaagcagggttgtcattgcaacaagcgacatgtccttaacaaatggtttggttgatgatacttacatggttcaaaatttgaaccacagagatagcttacaactatttcactatcatgcctttattgatgatcaagccaatcctcaaaagaaagatttcatgaagctgtcagaagggtttgtacattacgccagaggccatccactagccctcaaagtattgggtggagagcttaataagaaaagtatggatcattggaattcgaaaatgaagaaacttgcacagagccccagccctaatattgtcagtgtcttccaagtgagttatgatgaattgactacagcgcagaaagatgcatttctcgacatagcctgtttcagatcacaggacaaggattatgtagaaagtctactggcttcatctgaccttggttcggctgaagcaatgagtgcagtcaaatctctcacggataagttcctgattaatacttgtgatggccgagtggagatgcatgatctattatataaattttcaagggaagttgatctgaaggcatctaatcaggatggtagcagacaacggaggctgtggctccatcaacacataatcaagggaggcataatcaatgtattgcaaaataaaatgaaagctgccaatgttagaggtattttcctagacttgtctgaagtggaagacgaaacgagcttagaccgcgaccacttcataaatatgggaaatctccggtatctcaagttctacaattcccattgtccacaggaatgtaaaaccaacaataaaatcaacatccctgataaacttaagctaccattgaaagaagttcgatgcctccactggctgaaattcccattggagacacttccaaacgatttcaacccaattaatcttgtcgaccttaagctgccctacagtgaaatggaacaactttgggagggtgacaaggacacaccatgcttaaggtgggtcgatctcaatcactcaagtaagttgtgcagcttgtcagggttatcaaaagctgaaaagcttcaaaggttgaaccttgaaggttgcacaacactgaaagcgtttccacatgatatgaaaaaaatgaaaatgcttgctttcttgaatctgaagggatgtacaagtctcgaatctcttccagagatgaatttgatttctctgaaaacacttactctcagcggctgctcaacttttaaggaatttccgttgatttcagataatatagaaactctatacttagatggcacagcaataagtcagcttcctatgaacatggagaagcttcagagacttgttgtattgaatatgaaagactgcaaaatgctagaggaaattccaggccgtgttggtgagctgaaagctcttcaagaactgatactctctgattgtttaaatctcaagattttcccagaaatcaacattagctttttaaacattttacttttggatgggacagccattgaagtgatgccacaattaccctcagtgcagtatttgtgcttaagcagaaatgctaagatcagctgccttcctgttggaatcagtcagctttctcaacttaaatggctggacctgaagtattgtacgagtcttacatcagttccagagtttccaccaaatcttcagtgcttagatgcacacggctgtagttcgctgaagacagtttcaaagcctttggcccgtatcatgccaactgaacagaatcattccacattcattttcaccaactgtgagaacctggaacaagctgcaaaggaggaaatcacatcgtatgctcaaaggaaatgccagctgttatcatatgctcggaaacgctacaatgggggtcttgtttcagaatccttgttcagcacttgctttcctggatgtgaagtgccttcttggttttgtcatgaaacagttggatctgagttagaagtaaaactcctcccacattggcatgacaagaagcttgccgggattgctctatgtgctgtagtctcatgtcttgacccccaagatcaagtcagccgcttgtcagtgacctgcacctttaaagtaaaagatgaagacaagtcttgggtcccatatacttgtccagtaggaagttggaccagacatggaggcgggaaagataaaattgagctagaccatgtcttcattggatacaccagttgcccacatactataaaatgtcatgaagaaggtaactcagatgaatgcaatcctaccgaagcctctcttaaatttactgtgacaggaggtacaagtgagaatggaaaatacaaggtgttgaagtgtggtttaagtttggtgtatgctaaggataaagacaaaaatagtgctcttgaaacaaagtacgatatgcttattggaaagagctttcaagaaacttcagaaggggttgatggaagggtaaagaaaacgaaaggaaagtacgttatgcctgttgaaaagaactttcaagaaactacagaaggggttgatggaagagtaaagaaaaagaaaaaaacaagaatggataatggacggccaaagaagaagcagagatcaggaagagatgataatcaaactaggatgcaagtggaactccaagaaggaaatataaattcagtgatcatgcacacggttaagaatttctga

SEQ ID NO: 6 - protein, 1217 aa, Arabidopsis thaliana No-0
METSSISTVEDKPPQHQVFINFRGADLRRRFVSHLVTALKLNNINVFIDDYEDRGQPLDVLLKRIEESKIVLAIFSGNYTESVWCVRELEKIKDCTDEGTLVAIPIFYKLEPSTVRDLKGKFGDRFRSMAKGDERKKKWKEAFNLIPNIMGITIDKKSVESEKVNEIVKAVKTALTGIPPEGSHNAVVGALGNSNAGTSSGDKKHETFGNEQRLKDLEEKLDRDKYKGTRIIGVVGMPGIGKTTLLKELYKTWQGKFSRHALIDQIRVKSKHLELDRLPQMVLGELSKLNNPHVDNLKDPYSQLHERKVLVVLDDVSKREQIDALREILDWIKEGKEGSRVVIATSDMSLTNGLVDDTYMVQNLNHRDSLQLFHYHAFIDDQANPQKKDFMKLSEGFVHYARGHPLALKVLGGELNKKSMDHWNSKMKKLAQSPSPNIVSVFQVSYDELTTAQKDAFLDIACFRSQDKDYVESLLASSDLGSAEAMSAVKSLTDKFLINTCDGRVEMHDLLYKFSREVDLKASNQDGSRQRRLWLHQHIIKGGIINVLQNKMKAANVRGIFLDLSEVEDETSLDRDHFINMGNLRYLKFYNSHCPQECKTNNKINIPDKLKLPLKEVRCLHWLKFPLETLPNDFNPINLVDLKLPYSEMEQLWEGDKDTPCLRWVDLNHSSKLCSLSGLSKAEKLQRLNLEGCTTLKAFPHDMKKMKMLAFLNLKGCTSLESLPEMNLISLKTLTLSGCSTFKEFPLISDNIETLYLDGTAISQLPMNMEKLQRLVVLNMKDCKMLEEIPGRVGELKALQELILSDCLNLKIFPEINISFLNILLLDGTAIEVMPQLPSVQYLCLSRNAKISCLPVGISQLSQLKWLDLKYCTSLTSVPEFPPNLQCLDAHGCSSLKTVSKPLARIMPTEQNHSTFIFTNCENLEQAAKEEITSYAQRKCQLLSYARKRYNGGLVSESLFSTCFPGCEVPSWFCHETVGSELEVKLLPHWHDKKLAGIALCAVVSCLDPQDQVSRLSVTCTFKVKDEDKSWVPYTCPVGSWTRHGGGKDKIELDHVFIGYTSCPHTIKCHEEGNSDECNPTEASLKFTVTGGTSENGKYKVLKCGLSLVYAKDKDKNSALETKYDMLIGKSFQETSEGVDGRVKKTKGKYVMPVEKNFQETTEGVDGRVKKKKKTRMDNGRPKKKQRSGRDDNQTRMQVELQEGNINSVIMHTVKNF

SEQ ID NO: 7 - DNA, 4137 bp, Arabidopsis thaliana No-0
  CDS (1)..(4134)
atgaccaattgtgaaaaggatgaggaattcgtgtgcatcagctgcgtagaagaggtacggtactctttcgtgagccacctctctgaagctctccgtcgaaaaggcataaataatgtggtcgtagatgtagatatcgatgatctgcttttcaaggagtctcaggcaaagatcgagaaagctggggtttctgtgatggttttacccggaaactgtgacccttccgaggtatggcttgacaagttcgccaaggttctcgagtgccaaaggaacaacaaggaccaggcggtggtttcagtgttgtacggtgacagtctattacgggaccaatggcttagcgagctggatttcagaggcttatcacgaattcaccaatccaggaaggaatgtagtgactctatacttgtagaagagattgtgagagatgtgtacgagacgcacttttatgttggacgaattggaatctattcgaagctgctggagattgaaaacatggttaacaagcaaccgataggcatccgttgtgttggaatttggggtatgcctggcataggaaagacaacacttgctaaagcagtctttgaccaaatgtctagcgcctttgatgcttcttgttttatcgaagactatgacaaatcaattcatgagaagggtctttattgtttgctggaggaacaacttttgccgggtaatgatgcaaccattatgaaactgagctcgctcagagacagattgaacagtaagagagttcttgttgttctcgatgacgtgtgcaatgctctggttgcagagtcttttctcgaggggtttgactggctaggacccggaagcctgatcatcataacctctagagataaacaagtgtttcgcctttgcggaatcaatcaaatatatgaggtccagggtttaaatgagaaagaggctcgtcaacttttcttgctgtctgcgtctataatggaggatatgggagagcagaatctccatgagttgtcagtgagagtaataagttacgctaatggaaacccgttagctatcagtgtttatggaagagagctgaaaggtaagaaaaaactctcagaaatggagactgcattcctcaaactcaagcgacgtcctccatttaagattgtcgatgcatttaaaagcagctacgacacactcagtgacaacgaaaagaacatttttttggacatagcttgttttttccagggagaaaatgtcaactacgtgatacaactgcttgagggttgtggtttctttccacatgttgaaattgatgtccttgttgacaagtgtctggtgactatttcagaaaaccgagtttggttgcataagctgacccaggatatcggccgagaaatcataaatggagaaacagtacagatcgagaggcgcagaagactgtgggaaccttggagcatcaaatatttattagaatataatgaacacaaagcaaatggagaacctaaaacaaccttcaaacgtgctcagggctctgaagagatcgaaggcctgtttctagacacatcaaacttaagatttgatctgcagccctctgcctttaagaatatgttgaaccttagattgctcaaaatttattgttccaatcctgaagtccatcctgtaatcaatttcccaacaggctctctgcattctcttcctaatgagctaagactcctccattgggagaactatcctctgaaatctttgcctcagaattttgatcctaggcaccttgtcgaaatcaacatgccgtatagtcaacttcagaaactttggggtggaaccaagaacctggagatgttgaggacgatcaggctttgccattcccagcatctagttgatatcgatgatctcttaaaagctgaaaatcttgaggtaattgatctccaaggttgtacgagactgcagaatttcccagccgcaggtcgattgctacgtctacgagttgtaaatctctcaggttgcataaagattaaaagtgtcctagaaattccaccaaatattgagaaactacatctacagggaactggcatattagcattaccagtttccactgttaagccaaaccatagagagcttgtgaattttctaacagaaattccgggtctttcagaggcctcgaaacttgagcgtttaacaagtctgctggaatctaactcatcttgtcaagatcttgggaagcttatttgcttggagctgaaagattgctcttgtttgcagagtctgccaaacatggctaatttagatcttaatgttcttgatctctcgggttgctcaagtcttaattctattcagggtttccctcgttttctgaaacagttatatcttggtggcactgcaataagagaagtgccacaacttcctcaaagtctagaaatcttgaatgcacatggatcttgtttgcgaagtctgccaaacatggctaatttagaatttctcaaagttcttgatctctctggttgctcagagctcgagactattcagggttttcctcggaacctaaaagagttatattttgctggcactacgttaagagaagtgccccaacttcctttaagcctagaggtcttgaatgcacatggttctgactcggagaagcttcctatgcattacaagttcaacaattttttcgatctatctcaacaagtggtcaacgattttttcttgaaagcgctgacttatgtaaaacacataccaagagggtatacgcaggaactcatcaacaaagctccgactttcagcttcagtgcgccctcacatacaaatcaaaacgccacatttgatctgcaaccaggatcttctgtaatgacacgactaaatcattcatggaggaacacgcttgtgggatttggtatgctggtggaagttgcatttcccgaggactactgtgatgctacagatgttggcataagttgtgtttgcagatggagcaacaaagaaggccgctcttgtaggatagaaagaaattttcattgttgggcaccagggaaagttgttccaaaagttcgaaaggatcatacgtttgtctttagtgatgtcaacatgcgcccaagtaccggtgaaggaaatgaccctgatatctgggctggattagttgtatttgagttctttcctatcaatcagcagacaaagtgtctaaatgataggttcacagtgacaagatgtggagtccgtgtaataaatgttgcaactggcaatacaagtcttgagaacatatcactagttttgtctttggatccagtggaggtttctggttatgaagtattgagagtcagctatgatgatttacaggagatggataaagttctatttctttacatagcgtctttgttcaatgacgaggatgttgattttgtggcaccacttattgccggtattgacttggatgttagctctgggctcaaggtcttagccgatgtgtctctcataagtgtatcatccaatggggaaatagtgatgcatagtttgcaaagacaaatgggcaaagaaatcctccatggacaatccatgctgctgtctgattgtgagagttccatgaccgagaatttgtctgacgtaccaaaaaaggagaagaaacatcgcgaaagtaaggtaaagaaagtggtttccataccggctatagacgagggagatctatggacttggcgaaagtacggtcaaaaagacatcttaggttctcgttttccaaggggttactacaggtgcgcttacaagttcacgcatggttgtaaagctacaaaacaagtccaacggagcgagaccgattcaaacatgttagctattacttacctatctgagcataaccatccacggcccactaaacgcaaggctctcgctgactccactcgttccacttcctcctccatctgctcagccataactacctctgcctcatctagagtcttccaaaacaaagacgaaccaaatcaaccccacttgccttcctcctccactcctcctagaaacgcggctgtcttgtttaaaatgacggacatggaggagtttcaggacaatatggaggtggataatgacgtcgtagatacacgtacactggcattgtttccagagtttcaacatcagccggaggaagaagacccatggtcaacattcttcgatgattataatttttacttttga

SEQ ID NO: 8 - protein, 1378 aa, Arabidopsis thaliana No-0
MTNCEKDEEFVCISCVEEVRYSFVSHLSEALRRKGINNVVVDVDIDDLLFKESQAKIEKAGVSVMVLPGNCDPSEVWLDKFAKVLECQRNNKDQAVVSVLYGDSLLRDQWLSELDFRGLSRIHQSRKECSDSILVEEIVRDVYETHFYVGRIGIYSKLLEIENMVNKQPIGIRCVGIWGMPGIGKTTLAKAVFDQMSSAFDASCFIEDYDKSIHEKGLYCLLEEQLLPGNDATIMKLSSLRDRLNSKRVLVVLDDVCNALVAESFLEGFDWLGPGSLIIITSRDKQVFRLCGINQIYEVQGLNEKEARQLFLLSASIMEDMGEQNLHELSVRVISYANGNPLAISVYGRELKGKKKLSEMETAFLKLKRRPPFKIVDAFKSSYDTLSDNEKNIFLDIACFFQGENVNYVIQLLEGCGFFPHVEIDVLVDKCLVTISENRVWLHKLTQDIGREIINGETVQIERRRRLWEPWSIKYLLEYNEHKANGEPKTTFKRAQGSEEIEGLFLDTSNLRFDLQPSAFKNMLNLRLLKIYCSNPEVHPVINFPTGSLHSLPNELRLLHWENYPLKSLPQNFDPRHLVEINMPYSQLQKLWGGTKNLEMLRTIRLCHSQHLVDIDDLLKAENLEVIDLQGCTRLQNFPAAGRLLRLRVVNLSGCIKIKSVLEIPPNIEKLHLQGTGILALPVSTVKPNHRELVNFLTEIPGLSEASKLERLTSLLESNSSCQDLGKLICLELKDCSCLQSLPNMANLDLNVLDLSGCSSLNSIQGFPRFLKQLYLGGTAIREVPQLPQSLEILNAHGSCLRSLPNMANLEFLKVLDLSGCSELETIQGFPRNLKELYFAGTTLREVPQLPLSLEVLNAHGSDSEKLPMHYKFNNFFDLSQQVVNDFFLKALTYVKHIPRGYTQELINKAPTFSFSAPSHTNQNATFDLQPGSSVMTRLNHSWRNTLVGFGMLVEVAFPEDYCDATDVGISCVCRWSNKEGRSCRIERNFHCWAPGKVVPKVRKDHTFVFSDVNMRPSTGEGNDPDIWAGLVVFEFFPINQQTKCLNDRFTVTRCGVRVINVATGNTSLENISLVLSLDPVEVSGYEVLRVSYDDLQEMDKVLFLYIASLFNDEDVDFVAPLIAGIDLDVSSGLKVLADVSLISVSSNGEIVMHSLQRQMGKEILHGQSMLLSDCESSMTENLSDVPKKEKKHRESKVKKVVSIPAIDEGDLWTWRKYGQKDILGSRFPRGYYRCAYKFTHGCKATKQVQRSETDSNMLAITYLSEHNHPRPTKRKALADSTRSTSSSICSAITTSASSRVFQNKDEPNQPHLPSSSTPPRNAAVLFKMTDMEEFQDNMEVDNDVVDTRTLALFPEFQHQPEEEDPWSTFFDDYNFYF

SEQ ID NO: 9 - DNA, 3654 bp, Arabidopsis thaliana Nd-1; identical to SEQ ID NO: 5
  CDS (1)..(3651)

SEQ ID NO: 10 - protein, 1217 aa, Arabidopsis thaliana Nd-1; identical to SEQ ID NO: 6

SEQ ID NO: 11 - DNA, 4137 bp, Arabidopsis thaliana Nd-1; identical to SEQ ID NO: 7
  CDS (1)..(4134)

SEQ ID NO: 12 - protein, 1378 aa, Arabidopsis thaliana Nd-1; identical to SEQ ID NO: 8